(19) United States
(12) Patent Application Publication
(10) Pub. No.: US 2002/0028469 A1
(43) Pub. Date: Mar. 7, 2002

(54) Method of defining genus of chemical compound and method of designing molecules

(76) Inventors: Ronald M. Burch, Wilton, CT (US); Richard S. Sackler, Greenwich, CT (US)

Correspondence Address: DAVIDSON, DAVIDSON & KAPPEL, LLC, 485 SEVENTH AVENUE, 14TH FLOOR, NEW YORK, NY, 10018, US

(21) Appl. No.: 09/963,232

(22) Filed: Sep. 26, 2001

Related U.S. Application Data
(63) Continuation of application No. 09/191,780, filed on Nov. 13, 1998.
(60) Provisional application No. 60/065,716, filed on Nov. 14, 1997.

(51) Int. Cl. G01N 33/53; C12P 21/04

(52) U.S. Cl. 435/7.1; 435/70.21

(57) ABSTRACT

In accordance with an embodiment of the present invention, a method is provided for defining the portion of one or more chemical compounds having binding affinity for a target receptor. One or more chemical compounds to be tested are identified and then one or more key component fragments of the compound(s) are identified (e.g., a compound that “generically” defines the surface conformation and surface charge density of the one or more chemical compounds is “designed”) which may impart affinity for the target receptor. Analogs containing one or more of the key component fragments are then identified or synthesized, and the analogs are coupled to a carrier to construct an analog-carrier conjugate. The analogs contain one or more functional groups such as carboxyl, hydroxyl, keto, amino, nitro, or sulfhydryl to react with the carrier molecule. Next, the analog-carrier conjugate is utilized to generate a panel of monoclonal antibodies in vivo or in vitro, wherein the monoclonal antibodies are capable of defining the characteristics of the key component fragments. Next, the monoclonal antibodies are assayed to determine which are most specific for the key component fragments of the chemical compound(s) and which bind to the chemical compound(s). Competitive binding assays, or other assays are then preferably conducted to determine the ability of the monoclonal antibodies to discriminate between different chemical compounds.

This application is a continuation of a provisional U.S. patent application No. 60/065,716, filed on Nov. 14, 1997, hereby incorporated by reference.

### BACKGROUND OF THE INVENTION

The process of discovering novel chemical entities begins with the identification of an appropriate biological target molecule that is deemed to play a pivotal role in the pathophysiology of a given disease. These target molecules are most typically known as enzymes or receptors. Their identity can be known explicitly at the primary amino acid sequence level, or may only be known to exist via certain experimental observables.

A major goal in both the pharmaceutical and agricultural industries is to discover novel ligands that are able to bind with high affinity and specificity to these biological target molecules. Depending on the therapeutic indication and the nature of the target molecule, molecules that can either stimulate or inhibit the native action of the target are highly sought. In this regard, many different approaches toward the discovery of new chemical entities have been developed. Typically, the approach(es) taken depend upon the information available regarding the target.

When the three dimensional structure of the target protein is known, it is possible to apply certain computational procedures which apply the three dimensional steric and electrostatic constraints of the binding site as a basis, toward an electronic search of large and diverse chemical structure databases. Putative ligands are selected from the data and are scored on the basis of their “goodness of fit” to the binding site. Algorithms for this technique are widely known and used, and include MCSS/hook, dock leapfrog, and receptor. Although there are reports of success, the approach is limited in that true ligand/receptor flexibility is not accounted for, explicit water molecules are not included in the procedure and the energy functions used to calculate thermodynamic parameters and docking scores are overly simplified. Compounding these problems are the clock speeds of modem computers' microprocessors which are many orders of magnitude too slow.

The three dimensional structure of a target protein may not known experimentally. In these situations, a database of knowledge may be available for the target protein which correlates the activity of a given ligand with variations in its structure. This is commonly known as a structure-activity relationship, (SAR). There are many molecular parameters that can be considered a part of an SAR including surface area, molecular weight, hydrophobicity, and molecular field. Metrics which describe some combination of these parameters can be correlated to desired biological activity. Subsequently, extrapolations beyond the initial SAR test set of molecules can lead to the discovery of new chemical entities. See F Y Wiselogle, ed. A Survey of Antimalarial Drugs, Ann Arbor: J W Edwards, 1946; P B Marshall; Some chemical and physical properties associated with histamine antagonism; Br. J. Pharmacol. 10:270-280, 1955; R P Stephenson, A modification of receptor theory, Br. J. Pharmacol. 11:379-384, 1965; and McConnell H M, Owicki J C, Parce J W, Miller D L, Baxter G T, Wada H G, Pitchford S., The cytosensor microphysiometer: biological applications of silicon technology, Science 257:1906-12, 1992, hereby incorporated by reference.

In certain circumstances, particularly when a target has only recently been discovered, the target cannot be obtained in crystalline form, negating the opportunity for structure-based approaches to new chemical entities. Likewise, an SAR is not always available to use as a basis for the discovery of new chemical entities. In this scenario, a typical approach toward the discovery of new chemical entities is to resort to screening methods. The best approach in this case is to assay a large number of structurally diverse molecules in search of a lead compound with the desired biological properties. Recent advances in high throughput, parallel synthesis are currently being used to generate extensive libraries of molecules for this very purpose. See for example U.S. Pat. Nos. 5,510,240, 5,593,853, 5,780,603, 5,525,735, and 5,359,115, hereby incorporated by reference. As an alternative source of compounds for screening, natural product extracts or fermentation broths can be used. However, these mixtures of compounds are known to be extraordinarily difficult to subdivide and purify down to a single active species.

The assay itself can also be a limitation in the search for new chemical entities. Frequently, the prerequisite reagents needed are rare or, prohibitively expensive, or otherwise unavailable. Moreover, the purity of the target protein may be inadequate for the purposes of a quantitative assay.

As an alternative to biological assay methods, it is known in the art that antibodies can be generated which can bind biological molecules or small molecule haptens. It is further known that antibodies can function as enzyme mimics by performing catalysis if the antibody is generated with specificity for a transition state analog.

The development of monoclonal antibody technology, first disclosed by Kohler and Milstein (Kohler, G. and Milstein, C., Nature, 256:495-497, 1975), and incorporated by reference herein, has allowed the generation of unlimited quantities of antibodies of precise and reproducible specificity. If a specific antigen from one animal is injected as an immunogen into a suitable second animal, the injected antigen will elicit an immune response. The Kohler and Milstein procedure involves the fusion of spleen cells obtained from the immunized animal, with an immortal myeloma cell line to produce hybridomas. Clones which produce an antibody having the requisite specificity are then selected from these hybridomas. The hybridomas produce monoclonal antibodies which are uniform in their properties and specificity.

The basic unit of immunoglobulin structure is a complex of four polypeptides—two identical low molecular weight (“Light”) chains and two identical high molecular weight (“heavy”) chains, linked together by both noncovalent associations and by disulfide bonds. The immunoglobulin unit may be represented schematically as a “Y”. Each branch of the “Y” is formed by the amino terminal portion of a heavy chain and an associated light chain. The base of the “Y” is formed by the carboxyl terminal portions of the two heavy chains. The carboxyl terminal portion of the heavy and light chains form the constant domains. The node of the “Y” is the so-called hinge region, and is quite flexible.

Each light and heavy chain of an antibody has a variable region at its amino terminus. The variable region of a light chain (V_{L}) is approximately 110 amino acids in length, and is distinct for each antibody. The variable region of a heavy chain (V_{H}) is also approximately 110 amino acids in length. Each light and heavy chain of an antibody also has a “constant region.” For the light chain, this constant region (C_{L}) is located at its carboxyl terminus, and is approximately 110 amino acids in length. The constant region for the heavy chain (C_{H}) is composed of multiple domains, each about 110 amino acids in length, the number depending upon the particular antibody class. The constant region domains are named C_{H}1, C_{H}2, and C_{H}3. The C_{H}1 region is separated from the C_{H}2 region by a hinge region which allows flexibility in the antibody. The variable region of each light chain aligns with the variable region of each heavy chain, and the constant region of each light chain aligns with the first constant region of each heavy chain.

The C_{H}2-C_{H}3 domains of the constant region of a heavy chain form an “F_{c} region” which is responsible for other biological properties of the immunoglobulin molecule, such as complement binding and binding to the F_{C} receptors expressed by lymphocytes, granulocytes, monocyte lineage cells, killer cells, mast cells and other immune effector cells.

Diversity in the variable regions of both the light and heavy chains is restricted to three “hypervariable” regions or complementarity-determining regions (“CDRs”), each of which contains from about five to about ten amino acids, or up to about 20 amino acids. The three CDRs of the variable region of each light chain form loops which are clustered together and are connected to the four remaining parts of the variable regions, called the framework regions (“FRs”) which are relatively constant. Antibody diversity is created by changing only the sequences of the CDRs. Thus, the variable region is composed of hypervariable loops attached to a rigid framework. A more detailed account of the structure of immunoglobulins can be found in Sequences of Proteins of Immunological Interest, 5th Edition, E. A. Kabat, T. T. Wu, H. M. Perry, K. S. Gottesman, and C. Foeller, NIH Publication No. 91-3242, herein incorporated by reference.

The CDRs are positioned on the antibody structure such that they describe a surface whose three-dimensional contour, hydrophobicity and surface charge density provide binding regions with specificity for the immunogen. The CDRs of antibodies generated in response to immunization with a hapten provide three-dimensional grooves which accommodate a hapten and exhibit specific binding for the hapten molecule.

### OBJECTS AND SUMMARY OF THE INVENTION

It is an object of the invention to provide a method of defining a genus of chemical compounds by providing antibodies useful for defining the surface conformation and charge of a small organic molecule, inorganic molecule, biological molecule or molecule class.

It is a further object of the invention to provide a method of modeling ligand-receptor interactions using antibodies with specificity for a ligand structure that directly reflects the solution conformation adopted by a ligand.

It also is an object of the invention to provide a means of determining the binding characteristics of ligands to the active site of a target receptor or enzyme, and for designing ligands that will bind to the active site of a target receptor or enzyme.

It is a further object of the present invention to provide a method of determining whether a compound to be tested has the same key component fragments (e.g., belongs to the same genus of compounds as a compound that generically defines the surface conformation and surface charge density of one or more compounds that have affinity for a target receptor).

It is a further object of the invention to generate monoclonal antibodies that discriminate in binding between a hapten molecule (created from a compound having desired binding characteristics) and compounds of similar structure.

An object of the invention is to increase the sensitivity and specificity of high-throughput screening.

It is an object of the invention to provide a method of high-throughput screening that is able to select out components from a solution that 1) have similar molecular and structural characteristics as that of a ligand (e.g., chemical compound) having affinity to a target receptor or enzyme; or 2) that have desired binding characteristics to a target receptor or enzyme.

Further, it is an object of the present invention to provide a means for high-throughput screening that overcomes the difficulties in identification, availability, or purification of receptors for uses in high-throughput screening, and that provides a more inexpensive means of screening for compounds that bind to the aforementioned receptors.

In accordance with an embodiment of the present invention, a method is provided for defining the portion of one or more chemical compounds having binding affinity for a target receptor. One or more chemical compounds to be tested are identified and then one or more key component fragments of the compound(s) are identified (e.g., a compound that “generically” defines the surface conformation and surface charge density of the one or more chemical compounds is “designed”) which may impart affinity for the target receptor. Analogs containing one or more of the key component fragments (e.g., that generically define the surface conformation and surface charge density of the chemical compound(s)) are then identified or synthesized, and the analogs are coupled to a carrier to construct an analog-carrier conjugate. The analogs contain one or more functional groups such as carboxyl, hydroxyl, keto, amino, nitro, or sulfhydryl to react with the carrier molecule. Next, the analog-carrier conjugate is utilized to generate a panel of monoclonal antibodies in vivo or in vitro, wherein the monoclonal antibodies are capable of defining the characteristics of the key component fragments (e.g., generically define the exterior molecular characteristics of the compound that generically defines the surface conformation and surface charge density of the chemical compound(s)). Next, the monoclonal antibodies are assayed to determine which are most specific for the key component fragments of the chemical compound(s) and which bind to the chemical compound(s). Competitive binding assays, or other assays are then preferably conducted to determine the ability of the monoclonal antibodies to discriminate between different chemical compounds.

In a preferred embodiment, a panel of antibodies is used and comprises at least two antibodies. In a more preferred embodiment, the panel is comprised of between 2-3 antibodies.

Preferably, the ability of the antibodies to mimic the binding site of a target receptor or to act as a receptor surrogate is determined by comparing the ability of the antibodies to bind a compound with known affinity for a target receptor. The antibodies provide an advantage over the use of natural receptors for models of drug binding, because the specificity and affinity of the antibodies for ligand binding can be modulated. Antibodies can be chosen for screening or modeling functions on the basis of their ligand binding specificities, which provides for the controlled variability of binding affinity. For instance, the antibodies can be used to select for compounds possessing a similar structure or activity to a drug molecule, or to select for compounds similar to a drug molecule substituted with different moieties. The antibodies of the present invention can be used to selectively screen for drugs which bind to different target receptors, or different receptor subtypes. For example, the antibodies may be optimized to bind compounds which selectively inhibit PDEIV over PDEIII.

In another preferred embodiment, two or more analogs are utilized to generate a panel of monoclonal antibodies such that each analog-carrier conjugate defines a portion of the small organic molecule, inorganic molecule, biological molecule or molecule class. The analog-carrier conjugates together may define the entire surface conformation of the small organic molecule, inorganic molecule, biological molecule or molecule class or may define a portion of the entire surface conformation of the small organic molecule, inorganic molecule, biological molecule or molecule class. In a more preferred embodiment, the dissociation constants for the binding of the monoclonal antibodies to each analog are measured, and those exhibiting the strongest binding are included in a panel such that each different analog is represented by monoclonal antibodies in the panel.

In accordance with a further embodiment of the present invention, a method of identifying compounds which have binding affinity for a target receptor. In this embodiment, monoclonal antibodies selected by methods of the embodiment discussed above are further utilized for screening candidate chemical compounds. The monoclonal antibodies are immobilized on a support and then a series of assays is conducted to screen one or more compounds of interest.

In accordance with a further embodiment of the present invention, a method of designing molecules possessing defined exterior molecular characteristics is provided. In this embodiment, monoclonal antibodies selected by methods of the embodiments discussed above are firther utilized for a method of designing molecules possessing defined exterior molecular characteristics. An organism is immunized with the monoclonal antibodies, or alternatively, the splenocytes from an organism can be immunized in vitro, to generate anti-idiotype monoclonal antibodies reactive against the antigen binding region of the immunizing monoclonal antibodies. The three-dimensional structure of the antigen binding region of the anti-idiotype antibodies is then determined and may, in preferred embodiments, be used as a guide in molecular modeling experiments.

In a further preferred embodiment of the method of designing molecules possessing defined exterior molecular characteristics, anti-anti-idiotypic antibodies are generated having binding specificity for the anti-idiotypic monoclonal antibody antigen binding region. In yet a further embodiment, the ability of the anti-anti-idiotypic monoclonal antibody to compete with the first monoclonal antibody for binding to the compound that generically defines the surface conformation and surface charge density of the small organic molecule, inorganic molecule, biological molecule or molecule class is determined.

For purposes of the present application, the term “carrier molecule” means a macromolecule which is large enough to be recognized by the immune system, and that allows a hapten molecule to be recognized by the immune system.

For purposes of the present application, the term “high-throughput screening” means a method of rapidly testing known or unknown samples for reactivity with or binding to antibodies or fragments of antibodies specifically designed by the method of the invention.

For purposes of the present application, the term “generically” means applying to the entire group or class.

For purposes of the present application, the term “idiotype” means the region of an antibody molecule that confers the binding ability for a specific antigen structure.

For purposes of the present application, the term “anti-idiotype antibody” means an antibody with binding specificity for the antigen binding region of another antibody.

For purposes of the present application, the term “antibody” means a protein from the family of immunoglobulins, or fragments of immunoglobulins.

For purposes of the present application, the term “ligand” means a substance that has the capacity for specific and noncovalent (reversible) binding to a target receptor.

For purposes of the present application, the term “target receptor” means a biomolecule such as an enzyme, cell surface receptor, nucleotide sequence, polysaccharide, lipid or protein, or a molecule not of biological origin that participates in chemical reactions or binding but “target receptor” should not construed to be limited to these examples.

For purposes of the present application, the term “hapten” means a small molecule that stimulates an immune response if the small molecule is attached to a macromolecular carrier so that it is large enough to be recognized by the immune system.

For purposes of the present application, the terms “small organic molecule, inorganic molecule, biological molecule or molecule class” or “small chemical compound” means a molecule or chemical compound of molecular weight less than about 1000 g/mole.

For purposes of the present application, the term “in vitro immunization” means the deliberate induction of an immune response in vitro.

For purposes of the present application, the term “in vivo immunization” means the deliberate induction of an immune response in vivo.

For purposes of the present application, the term “IC₅₀” means the concentration of a compound that inhibits 50% of the activity of an enzyme.

For purposes of the present application, the term “conjugate” means a molecule created by fusing together two different molecules by chemical or recombinant DNA means.

For purposes of the present application, the term “analog-carrier conjugate” means the conjugate created by covalently or noncovalently attaching the hapten molecule to the carrier molecule.

For purposes of the present application, the term “surface conformation” means a three-dimensional contour of a molecule, portion of a molecule, or group of molecules.

For purposes of the present application, the term “surface charge density” covers all aspects of association, including free energy of binding, solvation, hydrogen bonding, van der Waals forces, charge, shape, chirality, the spatial variation of charges, or partial charges, around the surface of a molecule, and the contributions of dipole moments.

For purposes of the present application, the term “epitope” means antigenic determinant.

For purposes of the present application, the term “CDR” means the complementarity determining region of an immunoglobulin.

For purposes of the present application, the term “antigen” means any molecule recognized by the immune system as foreign, or against which an immune response can be induced.

For purposes of the present application, the term “analog” means a chemical compound possessing a similar structure to a model compound of interest.

For purposes of the present application, the terms “affinity” or “binding affinity” mean the strength with which the antibody binds to a binding partner.

For purposes of the present application, the phrase “compound that generically defines” means a compound whose three-dimensional structure and molecular characteristics approximate the three-dimensional contour and molecular characteristics of another molecule or molecule class.

For purposes of the present invention, the phrase “key component fragment” means a portion of a molecule which potentially contributes to the binding affinity of that molecule for a target receptor.

For purposes of the present application, the phrase “monoclonal antibodies generated from these analogs define” means that the monoclonal antibodies provide a three-dimensional structure, one surface of which is complementary to the surface conformation and surface charge density of another molecule or portion of a molecule.

For purposes of the present application, the term “coupling” shall mean covalent or noncovalent attachment.

### BRIEF DESCRIPTION OF THE DRAWINGS

The following drawings are illustrative of embodiments of the invention and are not meant to limit the scope of the invention as encompassed by the claims.

FIG. 1 shows the organic molecule, Compound A, which exhibits PDEIV inhibitory activity. Two analogs, Compound B and Compound C, of this inhibitor are shown, illustrating the placement of free amino groups for covalent linkage to the protein keyhole limpet hemocyanin (KLH).

FIG. 2 illustrates the synthetic scheme for Compound B.

FIG. 3 illustrates the synthetic scheme for Compound C.

FIG. 4A shows the absorbance spectra of succinylated KLH.

FIG. 4B shows the absorbance spectra of an analog of the compound that generically defines the structure of a PDBIV inhibitor, Compound C.

FIG. 4C shows the absorbance spectra of an analog of the compound that generically defines the structure of a PDEIV inhibitor, Compound B.

FIG. 4D shows the absorbance spectra of Compound C conjugated to succinylated KLH.

FIG. 4E shows the absorbance spectra of Compound B conjugated to succinylated KLH.

### DETAILED DESCRIPTION

Compounds which may be used in conjunction with the methods of the present invention include organic molecules, inorganic molecules and biological molecules, all of which have affinity for a target receptor.

### Specific Compounds

In certain embodiments of the present invention, the compounds which bind the target receptors are small organic molecules or a class of small organic molecules, such as systemically active drugs. A representative but non-limiting list of such classes of organic molecules include anorexic drugs such as amphetamines, phentermine, phenylpropanolamine and phenmetrazine; antiasthma drugs such as terbutaline sulfate, isoetharine, theophylline, and particularly newer selective PDEIV inhibitors which are discussed in detail herein; anticonvulsant drugs such as clonazepam, valproic acid, phenytoin, diazepam and primidone; antidepressant drugs such as trimipramine maleate, and imipramine HCL; antidiabetic drugs such as glipizide, glyburide, chloropropamide, acetohexamide, tolbutamide and tolazamide; anti-gout drugs such as probenecid, sulfinpyrazone and allopurinol; antihistamine drugs such as triprolidine HCL, diphenhydramine HCL, chloropheiramine maleate, brompheniramine maleate and hydroxyzine HCL; antimigraine drugs such as ergotamine tartrate, propanolol HCL metoprolol, isometheptene and mucate; antineoplastic drugs such as paclitaxel, tamoxifen citrate, mitotane, megestrol acemelphalan, amsacrine, streptozocin, anthracyline agents, azacitidine, bleomycide, vinca allkaloids, cytrarabine, hexamethylmelaine, methotrexate, hydroxyaren, chlorotriansene, cisplatin, cyclophosphamide, decarbazine, dactinomycine, methramycine, mitomycino, procarbazine, azathioprine, mercaptopurine, thioguanine and nitrosouress; decongestant drugs such as phenylephnine HCL, ephedrine and phenylpropanolamine; diuretic drugs such as thiazides, acetazolamide, furosemide and triamterene; hormone drugs such as estrogen, progesterone, testosterone and their analogues and derivatines; muscle relaxant drugs such as dantrolene sodium, cyclobenzaprine, clorozoxazone and quinine sulfate; sedative drugs such as barbiturates, meprobromate, promethazine HCL and methaqualone; tranquilizer drugs such as diazepam, chloroazepate monopotassium, prazepam, chloridiazepoxide HCL and chloralhydrate; antilipemic agents such as niacin, lovastatin, clofibrate, colestipol HCL, dextrothyroxine sodium; antiparkinson agents such as benztropine mesylate, carbidopa, levodopa, seligiline, pergolide, procyclidine, trihexylphenidyl; corticosteroids such as prednisone, triamcinolone, methylprednisolone, dexamethasone, betamethasone; bronchodiliators such as albuterol, atropine, epinephrine, isoproterenol, terbutaline; calcium channel blockers such as diltiazem, nicardipine, nifedipine, felodipine, verapamil; nonsteroidal anti-inflammatory drugs (NSAIDs) such as ibuprofen, fenoprofen diclofenac, piroxicam, nabumetone and naproxen; opioid analgesics such as morphine, hydromorphone, oxycodone and codeine; N-methyl-D-aspartate (“NMDA”) antagonists, such as dextromethorphan, dextrorphan and ketamine; Cyclooxygenase (“COX”) I inhibitors such as celecoxib (SC-58635), DUP-697, flosulide (CGP-28238), meloxicam, Vioxx (L 745,337), 6-methoxy-2 naphthylacetic acid (6-MNA), MK-966, nabumetone, nimesulide, NS-398, SC-5766, SC-58215, T-614. Other drugs and classes of drugs are contemplated

With respect to opioid analgesic agents for example, in certain embodiments it is preferred that such agents having structurally similarity be considered as a subclass. For example, this would group the phanthrenes together (i.e., codeine, hydrocodone, hydromorphone, levorphanol, morphine, oxycodone and oxymorphone), the phenylpiperidines together (alfentanil, fentanyl, meperidine, sufentanil) and the diphenylheptanes together (methadone, propoxyphene).

Non-pharmaceutical agents may also be utilized in conjunction with the methods of the invention, including for example, agrichemicals such as herbicides including 2,4-D and its derivatives, class of nitrobenzene amines, prometrone, atrazine, simazine, tribluralin, picloram, lindane, batoxyethanolesters, dimethylamine, diquat silvex, tok, machete, lasso, avenge, prowl and their derivatives.

The methods of the invention are applicable to inorganic or bioinorganic compounds as well. Examples of inorganic complexes are cisplatin (diamminedichloroplatinum), cobalomin and Ru-bisbipyridine. Examples of bioinorganic complexes include the metalloporphyrins such as copper(II) meso-tetra (N-methyl-4-pyridyl)porphyrin, which has been shown to intercalate into DNA, Biochemistry 35, 2818-2823 (1996). Additional inorganic complexes which maybe utilized for the present invention are enantiomers of [Ru(phen)2dppz]2+, which are known to participate in enantioselective intercalation with DNA oligomers, Inorganic Chemistry 36, 33 (1997), or the compound Rh(phenanthroline-2-9,10-phenanthrenequinone-diimine) which has been reported to act as a shape selective probe of DNA triple helices, Biochemistry 37, 9138-9146 (1998).

The method of the invention can also be utilized to model the structures formed by metalloproteins or fragments of active sites of metalloproteins, such as the zinc finger motif of Zif268 in which the metal is chelated with histidine and cysteine side chains, Science 252, 809 (1991), or the 18 residue zinc finger motif from the gag protein p55 of HIV, Biochemistry 29, 329 (1990). The method may be utilized for additional DNA binding proteins, such as the GAL4 transcription activator, Nature 356, 6368 (1992), which forms a bimetal-thiolate cluster of zinc ions with cysteine residue side chains.

Examples of biological compounds, which may be used in conjunction with the methods of the present invention, include proteins, peptides, lipids and lipoproteins, sugars, nucleic acid polymers or oligomers, antisense DNA or RNA oligomers or peptides, triplex or tetraplex forming DNA or RNA polynucleotides or oligomers, disaccharides, trisaccharides and other polysaccharides, neuraminic acid and other sugar acids. The biological compounds may be modified proteins or peptides, where the modifications are post-translational modifications such as, but not limited to, proteolytic cleavage, glycosylation, acetylation, acylation, methylation, pegylation, phosphorylation or myristoylation. In some instances, the biological compounds may be modified by chemical reagents, such as amidation, derivatization with known protecting or blocking groups, or cross-linking to a cellular ligand or other protein or molecule, but are not limited to these examples. By way of illustration only, the biological compounds may be hormones, neurotransmitters, second messengers, cell surface receptors, DNA replication or transcription regulatory elements, and the like.

The chemical compounds of the present invention are generally small in size. In certain preferred embodiments, the molecules have a molecular weight of less that approximately 1000 g/mole. In other certain preferred embodiments, the molecules have a molecular weight of less that approximately 500 g/mole.

### Identification of Analogs Containing Key Component Fragments

Once a desired chemical compound or compounds having affinity for a target receptor are chosen, one or more key component fragments of the compound(s) are identified which may impart affinity for the target receptor (e.g. a compound that generically defines the surface conformation and surface charge density of the one or more chemical compounds having affinity to a target receptor is “designed”). Ideally, the sum total of the key component fragments generically define the surface conformation and surface charge density of the desired compounds. Additional compounds containing some or all of the key component fragments are then identified as analogs. The analogs can be identified through review of existing literature to identify structurally similar compounds. The analogs containing the key component fragments (e.g. that contain the surface conformation and surface charge density of the chemical compounds that impart affinity to the target receptor) also can be synthesized or designed.

### Coupling of Analog to Carrier Molecule

Once the analogs are identified, they are coupled to a carrier molecule. In order for the compound(s) and one or more analogs to be coupled, they must have at least one functional group to form a covalent linkage. Thus, in designing analog compounds, the location and suitability of points of attachment to a carrier molecule are identified at several different parts of the molecule or are created. In certain embodiments, the desired chemical compound(s) and the analogs are the same, i.e., the compound(s) already have the desired points of attachment.

Preferably, the desired chemical compounds and/or analogs contain functional groups such as carboxyl, hydroxyl, keto, amino, nitro, sulfhydryl to react with the carrier molecule The analogs containing the key component fragments (e.g. that define different portions or aspects of the structure of the compound that generically defines the surface conformation and surface charge density of compounds with binding affinity for a target receptor) are then coupled to a carrier to construct one or more analog-carrier conjugates, such that one or more of the key component fragments are exposed. When the analogs are coupled to a carrier molecule, such as KLH, an “analog-carrier conjugate” is created. This process, known as haptenization, results in the generation or production of monoclonal antibodies with specificity for the compound. The free end of the molecule which is exposed to the solution (the “solvent exposed portion”) is most likely to be recognized as a hapten by the immune system. Therefore antibodies will be generated which exhibit selectivity for the solvent exposed portion of the molecule. The sterically hindered portion of the analog does not elicit a specific antibody reaction because it is not accessible to the antibodies.

Appropriate carriers include, for example, KLH (Keyhole Limpet Hemocyanin), ovalbumin or thyroglobulin. In preferred embodiments, the coupling occurs by means of the crosslinking of free amino groups on the carrier molecule and the analog using carbodiimide. Even more preferably KLH is the carrier.

The coupling can occur via covalent and noncovalent binding, but are preferably covalent in nature. The coupling method discussed herein is not limited to carbodiimide coupling methods, but includes general coupling methods known in the art. In certain embodiments, the analog-carrier conjugate can be created through recombinant DNA methods, for example, through the use of a fusion protein.

Methods to effect hapten coupling are known in the art, for instance, the method of coupling okadaic acid to a carrier protein using an activated ester was described in U.S. Pat. No. 5,263,556, herein incorporated by reference. Additional methods and materials are available by referring to the Pierce Chemical Co. catalog of products, herein incorporated by reference, where a wide variety of chemical coupling and cross linking agents are commercially available. See also P. Tijssen Practice and Theory of Enzyme Immunoassays, New York: Elsevier, 1985; and Fluorescent Probes and Research Chemicals, Chapter 5, Crosslinking and photoreactive probes., Molecular Probes, Eugene OR, 1998, hereby incorporated by reference.

In certain preferred embodiments, a peptide or peptide analog of a desired region of a biological molecule is coupled to the carrier molecule to create a hapten. For example, the peptide sequence which constitutes the zinc finger motif is coupled to the carrier molecule to create a hapten. In additional embodiments, the polysaccharide or non-peptide mimetic of a desired region of a biological molecule is used to create a hapten. In further embodiments, an analog of a post-translationally modified biological molecule is used to create the hapten. The post-translational modifications of the biological molecule are myristoylation, glycosylation, acetylation, methylation, pegylation, phosphorylation, amidation, proteolytic cleavage, linkage to a cellular ligand or other protein, but are not limited to these examples. The biological molecule may be modified by derivatization with known protecting or blocking groups

### Generation of Monoclonal Antibodies

The analog-carrier conjugates constructed by the methods discussed above are then utilized to generate monoclonal antibodies in vitro or in vivo, such that the monoclonal antibodies are capable of defining the characteristics of the key component fragments (e.g. generically define the surface conformation and surface charge density of the one or more compounds having affinity for the target receptor) . In vitro immunization is preferred if the analog-carrier conjugates would be subject to metabolic decomposition, in vivo. For in vivo immunization, see Harlowe and Lane, Antibodies: A Laboratory Manual, Cold Spring Harbor Publications, N.Y. (1988) and P. Tijssen, Practice and Theory of enzyme Immunoassays, New York, Elsevier, 1985, hereby incorporated by reference. For in vitro immunization, see BD Boss, An improved in vitro immunization procedure for the production of monoclonal antibodies, Meth. Enzymol. 121:27-32, 1986, hereby incorporated by reference.

### In Vitro Immunization

An example of a suitable in vitro immunization method is as follows:

Groups of eight Balb/c mice are immunized with each conjugate, and boosted at the end of weeks three and six. Test bleeds are done from each mouse ten days following the second boost. Serum is assayed by ELISA, using both the conjugates and KLH alone. Animals that have adequate titers against the conjugate over KLH alone were boosted one more time, and fusion is done four days later. Fusions are done with splenocytes from the mice and a murine myeloma cell line using polyethylene glycol. Selection is done using HAT media. For positive hybridomas, cloning is done using limiting dilution. Following cloning, hybridomas are injected into nude mice to produce adequate quantities of antibodies, and antibodies are purified from ascites fluid.

Alternatively, the in vitro immunization can be conducted as follows:

The spleen is removed from a Balb/c mouse. Splenocytes are dissociated by trituration of the through a 20 g needle. Splenocytes are distributed to the wells of a 96-well plate containing a near-confluent layer of MR-5 fibroblasts. Antigen is added and the cultures are incubated for three days. Wells are visually examined for splenocyte proliferation and the contents of those wells are fused with a murine myeloma cell line using polyethylene glycol. Selection was done using HAT media. For positive hybridomas, cloning is done using limiting dilution. Following cloning, hybridomas are injected into nude mice to produce adequate quantities of antibodies, and antibodies are purified from ascites fluid.

### Panel of Antibodies

In a more preferred embodiment, a panel of monoclonal antibodies is created, wherein the various monoclonal antibodies chosen have affinities for one or more of the key component fragments (e.g. for different aspects of the compound that generically defines the surface conformation and surface charge density of the one or more chemical compounds having affinity for the target receptor). The antibody panel together provides a receptor binding site model, and can be used as a receptor surrogate for design and screening of molecules. Antibodies with specific binding for the key component fragments (e.g. the portion of the compound that generically defines the surface conformation and surface charge density of the one or more chemical compounds having affinity for the target receptor) together provide a partial receptor binding site model. In an even more preferred embodiment, the panel is comprised of at least two antibodies. In a most preferred embodiment, the panel is comprised of between 2-3 antibodies.

In another preferred embodiment, two or more analogs are utilized to generate the panel of monoclonal antibodies such that each analog-carrier conjugate defines a portion of the one or more chemical compounds having affinity for the target receptor. The analog-carrier conjugates together may define may define a portion of the entire surface conformation of the one or more chemical compounds having affinity for the target receptor, or may define the entire surface conformation of the one or more chemical compounds having affinity for the target receptor.

### Assaying Monoclonal Antibodies for Specificity

Next, the monoclonal antibodies are assayed to determine which are most specific for the key component fragments of the one or more chemical compounds and which bind to the one or more chemical compounds.

Monoclonal antibody binding assays are based on the noncovalent association of two molecules, or the principle of molecular recognition. The non-covalent association of any two molecules, regardless whether they are antibodies, peptides, proteins, or small molecules (organic or inorganic), in any combination, is governed by two main factors: shape complementarity and favorable electrostatic forces.

For example, shape complementarity requires that the sizes and shapes of the associating pairs match and is a further function of overall conformational preferences, molecular size, chirality, and solvation.

Electrostatic forces also must be complimentary between associating pairs, which is a function of the atomic composition, degree/distribution of lone electron pairs and unoccupied molecular orbitals. In addition, solvation is critical as the highly polarized water molecules can shield charges on certain functional groups or can promote hydrogen binding via complex solvation networks.

Molecular recognition can also be described in terms of the thermodynamic aspects of association, which is calculated using equations that summarize the contributions from each of their components. For a thorough treatment, a statistical mechanical treatment of flexibility of the molecules must also be considered. This is usually treated using an estimate of a thermodynamic value known as entropy (ΔS). Indeed, a typical equation from thermodynamics relates free energy (ΔG) with entropy (ΔS) as follows: ΔG=ΔH−TΔS. Note that ΔH is an enthalpic term, a T is temperature. As temperature increases, it clearly impacts free energy of association by increasing the contribution of the entropic term by forcing a given molecule to “populate” a larger number of discrete conformational states. Finally, ΔG can be related to an equilibrium constant as follows:

ΔG=−RT In K_{eq}

It should be noted that the inverse of an equilibrium constant is what is measured in a binding assay. Hence, these equations relate binding with structural flexibility (entropy), temperature, and enthalpy.

Thus molecular recognition in general involves shape complementarity, favorable electrostatic forces and a decrease in the free energy of association between the two molecules.

Recognition of antigen by an antibody is based on the same principles. Generally speaking, “. . . antibodies recognize the overall shape of an epitope rather than particular chemical residues. Antibodies are capable of expressing remarkable specificity, and are able to distinguish between small differences in . . . charge, optical configuration and steric conformation.” I Roitt, J Brostoff, D Male Immunology 5^{th} ed. London, Mosby, at p. 10 (1998), hereby incorporated by reference. For example, antiserum raised to the metaisomer of aminobenzene sulphonate is able to discriminate between the ortho and para isomers of aminobenzene sulphonate, and also between the three isomers (O−, m−, and p−) of aminobenzene arsonate and aminobenzene carboxylate. The antiserum reacts strongly with the sulphonate in the meta position, weakly with the sulphonate in the ortho position and only marginally with the sulphonate in the para position. The antiserm binds only weakly to the meta-arsonate which is larger (and not at all with the ortho or para arsonates) and not to the isomers of the carboxylate, which is considerably smaller than sulphonate. I Roitt, J Brostoff, D Male Immunology 5 ^{th} ed. London, Mosby, at p. 10 (1998), hereby incorporated by reference.

The surface area of contact between antigen and antibody can vary enormously among antibodies, from a few Angstroms to more than 1.5 nm² (Roitt as above, p. 112; D Wang, J Ligo, D Mitra, P Akolkar, F Gruezo, and B Kabat. The repertoire of antibodies to a single antigenic determinant. Mol. Immunol. 28:1387-1397, (1991). If the antigen is a macromolecule, the binding site is “planar,” while if the antigen is a bit smaller, the binding site is a cleft. For small antigens, the binding site is a small cavity.

The exterior molecular characteristics approximately represent the topological profile of the small molecule antigen against which the antibody was raised. This profile is a combination of 3-dimensional conformation, molecule structure and electrostatic charge distribution, and may also include some bound water molecules. These collective parts are what the antibody recognizes.

In other words, a small molecule (or any ligand) with a desirable binding affinity to a given target protein, can be used to generate antibodies with specificity for the topology of that ligand. The CDR regions of these antibodies structurally encode an imprint of the ligand topology. This is assumed to be maintained even in the absence of the ligand. It follows that the encoded topology in the CDR domain, should represent a similar structural and electrostatic environment as the ligand binding site on the target protein (usually an enzyme or receptor).

Compounds which bind to the target receptor have the exterior molecular characteristics (surface conformation and surface charge density) that result in a binding interaction with the target receptor. The key component fragments of the analogs are chosen to duplicate that binding interaction with the target receptor. Thus, the monoclonal antibodies which result from the immunization procedures above should generically define the surface conformation and surface density of the portion of the analog they are raised against (e.g. the one or more key component fragments).

In other words, the monoclonal antibodies generated from these analogs define the surface conformation of the compound that generically defines the surface conformation and surface charge density of the small organic molecule, inorganic molecule, biological molecule or molecule class. The relative affinity of the antibodies for different analogs of the compound that generically defines the surface conformation and surface charge density of the small organic molecule, inorganic molecule, biological molecule or molecule class provides a method of defining the genus of chemical compounds to which each tested compound belongs. Antibodies able to discriminate between compounds can be used to generate structure-activity relationships for design of additional compounds exhibiting the desired activity of the compound that generically defines the surface conformation and surface charge density of the small organic molecule, inorganic molecule, biological molecule or molecule class. These antibodies can also be used for quantitative analysis of binding affinity and theoretical modeling of additional candidate compounds. The antibodies may also be used to model compounds which selectively bind to additional receptor or enzyme subtypes.

### Exemplative Assays

Antibodies selected using the above-mentioned screening procedures are subjected to further testing in order to select antibodies possessing the desired specificity and binding affinity against particular drug moieties. The specificity and affinity of the antibodies are screened using methods known in the art. Examples of such assays include competitive binding assays, direct binding assays, immunochemical assays, saturation assays or standard assays such as ELISA, EIA, RIA, plasmon resonance spectrometry and flow cytometric assays. When the small organic molecule, inorganic molecule, biological molecule or molecule class has a chromophore, is fluorescent or can be tagged with a fluorophore, chromophore or radioisotope, a direct binding assay method is preferred. In some competition assays, the ability of the antibodies or antibody fragments to bind to an antigen is determined by detecting the ability of the antibodies or antibody fragments to compete with the binding of a compound or receptor known to bind to the antigen. Numerous types of competitive binding assays are known, for example, as discussed in U.S. Pat. No. 5,821,123, or Harlowe and Lane, Antibodies: A Laboratory Manual, Cold Spring Harbor Publications, N.Y. (1988), herein incorporated by reference.

Assays for measuring binding of a test compound to one component alone rather than using a competition assay are also available. For instance, antibodies may be used to identify the presence of a T cell marker. Standard procedures for monoclonal antibody assays, such as ELISA, may be used. For a review of various signal producing systems which may be used, see U.S. Pat. No. 4,391,904, incorporated by reference herein.

In a more preferred embodiment, the monoclonal antibodies generated for each analog are tested for their binding affinity, and those exhibiting the strongest binding are included in a panel such that each different analog is represented by monoclonal antibodies in the panel. In certain embodiments, the binding affinity of the monoclonal antibodies is in the range of from about 10⁸ to about 10^{11,} or the monoclonal antibodies have a K_{D} of from about 0.01 nM to about to 10 nM. Preferably, the K_{D} is approximately 1 nM. In even more preferred embodiments the K_{D} is less than approximately 1 nM.

The antibodies may include a complete antibody molecule having full length heavy and light chains, or any fragment thereof, such as the Fab or (Fab′)₂ fragments, a heavy chain and light chain dimer, or any minimal fragment thereof such as a Fv or a SCA (single chain antibody) or any other molecule with the same specificity as the antibody.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given monoclonal antibody may be determined according to well known methods. Those skilled in the art will be able to determine operative and optimal assay conditions for each determination by employing routine experimentation.

One of the ways in which the monoclonal antibody can be detectably labeled is by linking the same to an enzyme and use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller et al., 1978, .J Clin. Pathol. 31:507-520; Butler, 1981, Meth. Enzymol. 73:482-523; Maggio, E. (ed.), 1980, Enzyme Immunoassay, CRC Press, Boca Raton, Fla.,; Ishikawa et al., (eds.), 1981, Enzyme Immunoassay, Kgaku Shoin, Tokyo)). The enzyme which is bound to the monoclonal antibody will react with an appropriate substrate, preferably a chromogenic substrate, in such a manner as to produce a chemical moiety which can be detected, for example, by spectrophotometric, fluorimetric or by visual means. Enzymes which can be used to detectably label the monoclonal antibody include, but are not limited to, malate dehydrogenase, staphylococcal nuclease, delta-5-steroid isomerase, yeast alcohol dehydrogenase, alpha-glycerophosphatase, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards. Detection may also be accomplished using any of a variety of other immunoassays. For example, by radioactively labeling the monoclonal antibodies or fragments, it is possible to detect the target that the antibody was designed to bind through the use of a radioimmunoassay (RIA) (see, for example, Weintraub, 1986, Principles of Radioimmunoassays, Seventh Training Course on Radioligand Assay Techniques, The ws Endocrine Society). The radioactive isotope can be detected by such means as the use of a gamma counter or a scintillation counter or by autoradiography. It is also possible to label the monoclonal antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The monoclonal antibody can also be detectably labeled using, fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The monoclonal antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the monoclonal antibody of the present invention. Bioluminescence is a type of chemiluminescence found in biological systems, in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin. Any binding assay known in the art can be used to assess the binding between the monoclonal antibody and the particular molecule. These assays may also be performed to select antibodies that exhibit a higher affinity or specificity for the particular hapten or key component fragment.

For example, but not by way of limitation, binding of the monoclonal antibody to the particular molecule can be assayed using various immunoassays known in the art including but not limited to, competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In certain embodiments, antibody binding is detected by detecting a label on the primary antibody. In other embodiments, the primary antibody is detected by detecting binding of a secondary antibody or reagent to the primary antibody. In further embodiments, the secondary antibody is labelled. Many means are known in the art for detecting binding in an immunoassay and are within the scope of the present invention.

An in vitro assay system useful in assessing the binding of the monoclonal antibody to its target molecule is described below. Briefly, a reaction mixture of the monoclonal antibody and the test sample is incubated under conditions and for a time sufficient to allow the two components to interact with, e.g., bind to each other, thus forming a complex, which can be removed and/or detected in the reaction mixture. These assays can be conducted in a variety of ways. For example, one method to conduct such an assay would involve anchoring the monoclonal antibody or the test substance onto a solid phase and detecting the antibodies/molecule complexes anchored on the solid phase at the end of the reaction. In one embodiment of such a method, the monoclonal antibody may be labeled, either directly or indirectly, and the test sample is anchored onto a solid surface. In practice, microtiter plates may conveniently be utilized as the solid phase. The anchored component may be immobilized by non-covalent or covalent attachments. Non-covalent attachment may be accomplished by simply coating the solid surface with a solution of the test sample and drying.

In order to conduct the assay, the noninuobilized component is added to the coated component. After the reaction is complete, unreacted components are removed (e.g., by washing) under conditions such that any complexes formed will remain immobilized on the solid surface. The as detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface

Alternatively, a reaction can be conducted in a liquid phase, the reaction products separated from unreacted components, and complexes detected.

The monoclonal antibodies (or functionally active fragments thereof) useful in the present invention may, additionally, be employed histologically, as in immunofluorescence or immunoelectron microscopy, for in situ detection of the molecule. In situ detection may be accomplished by removing a histological specimen from an animal or plant, such as paraffin embedded sections of affected tissues and applying thereto a labeled monoclonal antibody of the present invention. The monoclonal antibody(or functionally active fragment thereof) may also be applied by overlaying the labeled antibody onto a biological sample. If the molecule to which the antibody binds is present in the cytoplasm, it may be desirable to introduce the monoclonal antibody inside the cell, for example, by making the cell membrane permeable. Through the use of such a procedure, it is possible to determine not only the presence of the particular molecule, but also its distribution in the examined tissue or bacterial colony, for example. Using the present invention, those of ordinary skill will readily perceive that any of a wide variety of histological methods (such as staining procedures) can be modified in order to achieve such in situ detection.

Immunoassays for the particular molecule will typically comprise incubating a sample, such as a biological fluid, a tissue extract, freshly harvested cells, or lysates of cultured cells, in the presence of a detectably labeled monoclonal antibody and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled monoclonal antibody. The solid phase support may then be washed with the buffer a second time to remove unbound antibody. The amount of bound label on solid support may then be detected by conventional means.

In preferred embodiments of the present invention, the monoclonal antibodies are used to verify that additional small molecules are part of the structural class to which the compound that generically defines the surface conformation and surface charge density belongs.

In a further preferred embodiment, the three-dimensional structure of the antigen binding region of the monoclonal antibody is determined. In yet a further preferred embodiment, the three-dimensional structure is used to design new molecules.

In more preferred embodiments, the three-dimensional structure of the antigen binding region is used to evaluate the exterior molecular characteristics of one or more compounds of interest to determine whether the compounds of interest have binding affinity at the target receptor.

The monoclonal antibodies, and preferably the panel of monoclonal antibodies, can also be utilized for various purposes including, inter alia: 1) modeling ligand-receptor interactions (e.g., interactions between a chemical compound and a target receptor) using antibodies with specificity for a ligand structure that directly reflect the solution conformation adopted by a ligand; 2) determining the binding characteristics of ligands (e.g., chemical compounds) to the active site of a target receptor or enzyme, and for designing ligands that will bind to the active site of a target receptor or enzyme; 3) the modeling of ligand-receptor interactions that include the solvated surfaces of the ligand and receptor binding sites and reflect the energetic contributions made to binding and biological activity; 4) determining whether a compound to be tested has the same key component fragments (e.g., belongs to the same genus of compounds as a compound that generically defines the surface conformation and surface charge density); 5) providing a surrogate binding partner for use in modeling experiments; 6) providing monoclonal antibodies that discriminate in binding between a hapten molecule (created from a compound having desired binding characteristics) and compounds of similar structure; 7) providing alternative ligand-receptor binding models useful for discriminating between compounds which bind with specificity to different receptor subtypes; and/or 8) providing a means of assessing the structure-activity relationship of compounds for a target receptor.

### High Throughput Screening

A further aspect of the invention is directed to a method of identifying compounds which have binding affinity for a target receptor. In this embodiment of the present invention, monoclonal antibodies selected by methods of the embodiment discussed above are further utilized for screening candidate chemical compounds. In a preferred embodiment, a high-throughput screening method is provided that is able to select out compounds from a solution that have similar molecular and structural characteristics to compounds that have affinity for a target receptor, such as a ligand to a target receptor or a compound that acts as a substrate for an enzyme. Preferably, the high-throughput screening method will select compounds that have desired binding characteristics to a target receptor or enzyme.

High throughput screening is a method of determining the potency of large numbers of receptor ligands or enzyme inhibitors. In a preferred method of the invention, the subject antibodies act as surrogate receptors. High throughput screening can be done using any of a large variety of techniques that can yield quantitative data. Methods such as dot blot assays and competitive inhibition by the drug candidates of binding of the generic drug-KLH preparation to the antibodies can be used. A high-throughput screening procedure that can be used with the monoclonal antibodies produced by the method of the invention is set forth below.

### Screening Compounds of Interest

A ligand labeled with a radioactive isotope or chromophore, for example, is added in identical concentration to each of multiple reaction vessels. The hapten used to generate the antibodies, or additional compounds known to bind to a receptor or enzyme, could be used. In some vessels, potential competing ligands are added along with the labeled ligand. The potency of a competing ligand is determined by observing how well a given concentration of competing ligand reduces binding of the labeled ligand, by competition for binding sites.

Reaction vessels can vary widely, and any vessels providing a convenient means of attaching antibodies to a surface is adequate. For example, the antibodies may be attached to the wells of multiwell microtiter plates. See, P Tijssen Practice and Theory of Enzyme Immunoassays, New York: Elsevier, 1985, Fluorescent Probes and Research Chemicals, Chapter 5, Crosslinking and photoreactive probes, Molecular Probes, Eugene Oreg., 1998, hereby incorporated by reference.

For an assay utilizing a microtiter plate, the labeled ligand and competing ligand are placed into each well. The plates are incubated for a time adequate for the labeled ligand to bind. The time required for binding can be determined empirically, and most often is 2-24 hours. Then, the plates are washed with additional media not containing ligands and the amount of bound label is quantitated. Quantitation is performed using liquid scintillation or solid phase fluorescence counting if a ligand labeled with a beta emitter is used. A gamma counter is used if the ligand is labeled with a gamma emitter. A microplate spectrometer is used if the ligand is labeled with a chromophore or fluorescent compound.

The percent inhibition of ligand binding by competitor is calculated by the following equation:

%I=1 −[(signal in absence of competitor−signal in presence of competitor)/signal in absence of competitor].

See, for example, Silverman L, Campbell R, Broach JR, New assay technologies for high-throughput screening, Curr. Opin. Chem Biol, Jun;2(3):397-403 (1998); Llewellyn LE, Doyle J, Negri A P, A high-throughput, microtiter plate assay for paralytic shellfish poisons using the saxitoxin-specific receptor, saxiphilin, Anal. Biochem, July 15;261(1):51-6 (1998); and Burbaum J J, Sigal N H, New technologies for high-throughput screening, Curr. Opin. Chem Biol., Jun;l (1):72-8 (1997), hereby incorporated by reference .

The means for high-throughput screening provided overcomes the difficulties in identification, availability, or purification of receptors for uses in high-throughput screening, and provides a more inexpensive means of screening for compounds that bind to the aforementioned receptors. The assays can be performed in a linear manner (i.e., sequentially) or in a parallel manner (simultaneously).

In certain embodiments, the antibody is adsorbed to a surface and used for screening, for example, using the microtiter assay described above. In other embodiments, the ligand can be immobilized on a support, for example, a bead, and fluorescently labeled antibodies can be allowed to contact and bind to the ligand. Competitive binding assays can be performed by introducing a compound which competes with the immobilized ligand for binding to the fluorescently labeled antibodies. Screening in this embodiment of the invention is performed using flow cytometry, as described, for example, in U.S. Pat. No. 5,510,240, herein incorporated by reference.

### Antibody Affinity Chromatography

In the present invention, the monoclonal antibodies generated from the analog-carrier conjugates may also be utilized to perform affinity chromatography. The antibodies are coupled to a chromatographic support medium using methods known in the art for constructing activated chromatographic support media for affinity purification. Coupling reactions for attaching the antibodies to the affinity support medium and elution conditions are likewise known in the art.

In a preferred method, an antibody affinity column is used as follows: 1) a solution of interest is allowed to contact the antibodies on the affinity support medium; 2) if molecules are present in the solution which possess the necessary conformation, solvated surface and charge characteristics, they will bind to the antibody composition; and 3) the retained compounds may thereafter be recovered by, e.g., eluting with a high salt content solution, changing the pH of the eluting buffer, or competitive displacement with compounds known to have a similar structure to the generic compound, small organic molecule, inorganic molecule, biological molecule or molecule class.

Compounds which are obtained on screening are tested for their ability to bind to the remaining members of the panel of antibodies. Compounds which bind to multiple members of the panel of antibodies are good candidates for further chemical compound research and development efforts. This screening procedure is generally applicable to screening efforts to identify molecules for purposes other than pharmaceutical discovery.

The mixture of compounds to be screened can be selected from, inter alia, any of the following: combinatorial libraries of peptides, synthetic ethers, carbohydrates, or phosphonates; natural product extracts; microbial or other cell culture broths; synthetic products; synthetic analogs; synthetic intermediates of drug candidates; or drug metabolites.

In a preferred embodiment, a method is provided of high-throughput screening that is able to select out components from a solution that 1) have similar molecular and structural characteristics as that of a ligand (e.g., chemical compound) having affinity to a target receptor or enzyme; or 2) that have desired binding characteristics to a target receptor or enzyme.

In another preferred embodiment a means for high-throughput screening is provided that overcomes the difficulties in identification, availability, or purification of receptors for uses in high-throughput screening, and that provides a more inexpensive means of screening for compounds that bind to the aforementioned receptors.

The high-throughput screening procedure allows verification that additional small molecules are part of the structural class to which a generic molecule belongs. Further, this embodiment allows for discrimination between the binding avidity of small molecules by the antibodies by using, for example, a salt or pH gradient, or a gradient of a competing ligand.

In a preferred embodiment, a small organic molecule is hooked to a protein (e.g., to make a hapten), and this composition is used to immunize an animal to produce antibodies recognizing the hapten. Antibodies of the required specificity and binding affinity are produced by conventional methods for generating monoclonal antibodies and the resulting antibodies are immobilized on an antibody affinity column. A solution is poured over the column, and if the appropriate antigen is present in the solution, it will bind to the composition. The bond may thereafter be broken by, e.g., pouring a high salt content solution over the column, allowing one to select out the component of the solution which is similar to the hapten used to generate the antibodies. This aspect of the invention allows verification that additional small molecules are part of the structural class to which a generic molecule belongs, and allows fractionation of a complex mixture of small molecules derived from libraries of synthetic ethers, natural product extracts, microbial or other cell culture broths, etc., to identify novel compounds that belong to the same class as a generic compound. This method would increase the sensitivity and specificity of high-throughput screening.

### Method of Designing Molecules

A further aspect of the invention is directed to a method of identifying compounds which have binding affinity for a target receptor. In this embodiment of the present invention, monoclonal antibodies selected by methods of the embodiments discussed above are further utilized for a method of designing molecules possessing defined exterior molecular characteristics.

In a further embodiment, if a specific antibody is used as an immunogen, the antibody itself will result in an immune response. Anti-antibodies are produced that are specific for the unique epitopes (idiotopes) of the variable domains of the injected antibodies; these epitopes are known collectively as the idiotype of the primary antibody and the secondary (anti-) antibodies which bind to these epitopes are known as anti-idiotypic antibodies. Other secondary antibodies will be specific for the epitopes of the constant domains of the injected antibodies and hence are known as anti-isotypic antibodies.

According to “network” theory, antibodies produced initially during an immune response will carry unique new epitopes to which the organism is not tolerant, and therefore will elicit production of secondary antibodies (Ab2) directed against the idiotypes of the primary antibodies (Ab1). These secondary antibodies likewise will have an idiotype, which will induce production of tertiary antibodies (Ab3), and so forth. The “network” theory also suggests that some of those secondary antibodies will have a binding site which is an analog of the original antigen, and thus will reproduce the “internal image” of the original antigen. In other words, an anti-idiotypic antibody may be a surrogate antigen.

The anti-idiotypic cascade can be understood as follows: an exogenous antibody “Ab1” to a cancer antigen is administered to a patient. That antibody, in addition to binding to its target antigen, serves as an antigen itself. Antibodies made to this foreign antibody are called anti-idiotypic antibodies. A subgroup of these anti-idiotypic antibodies recognizes as its antigen an actual CDR of Ab1. These antibodies thus tend to resemble the antigen to Ab1 in structure and are called internal image-bearing anti-idiotypic antibodies, or “Ab2.” In this cascade, Ab2 can in turn be recognized as an antigen, and anti-idiotypes to Ab2 are generated. A subset of these anti-idiotypes will also be internal image-bearing anti-idiotypes “Ab3” and hence their binding site resembles the binding sites of the antigen for Ab2, or Ab1. Anti-idiotypic antibodies thus have binding sites that are similar in conformation and charge to the original antigen or hapten.

For example, V. van Cleave and colleagues have immunized mice with a rabbit protein and were subsequently able to isolate two monoclonal antibodies that each contained a sequence identical to the rabbit antigen within CDR2 of their heavy chains (J. Exp. Med. 167:1841-1848, 1988). Thus, anti-idiotype antibodies were produced which duplicated the original antigen sequence. The synthetic peptides containing the CDR sequence of the anti-idiotype antibody competed with antibody specific for the rabbit protein.

W. V. Williams and colleagues further demonstrated that peptides prepared from the CDRs of anti-idiotype antibodies can compete with the complete antibody for antigen binding (Proc. Natl. Acad. Sci. USA 86:5537, 1989), herein incorporated by reference. In particular, Williams et al. investigated Monoclonal antibody 87.92.6, an anti-idiotype antibody generated against an antibody specific for reovirus type 3 hemagglutinin, binds to the same protein as does reovirus type 3 (the reovirus hemagglutinin) and competes with reovirus for binding at those specific proteins. A comparison of the sequences of the six CDRs of monoclonal antibody 87.92.6 with the sequence of reovirus hemagglutinin reveals that the amino acid sequence of CDR2 of the light chain of monoclonal antibody 87.92.6 is homologous to amino acids 317-332 of reovirus hemagglutinin. In other words, the CDR sequence with specific binding for the antibody to reovirus shows sequence homology to the sequence for reovirus hemagglutinin. Further, a peptide with the same sequence as the sequence of CDR2 of the light chain competes with the binding of reovirus hemagglutinin to reovirus receptor.

Anti-idiotype antibodies may also reproduce or mimic the structure of the antigen rather than the primary sequence itself. An illustration of the ability of an anti-idiotype antibody to mimic the structure of the primary antigen is provided by Monfardini et al., J. Biol. Chem. 270, 6628-6638 (1995) and Monfardini et al., Proc. Assn. Am. Physicians 108, 420-431 (1996), herein incorporated by reference. In these studies, anti-idiotype antibodies to anti-GM-CSF antibodies were generated. One of the anti-idiotype antibodies inhibited the binding of GM-CSF to the GM-CSF receptor. Analysis of the CDR region exhibiting the best fit to the putative GM-CSF binding region revealed that the anti-idiotype antibody CDR had only a weak sequence similarity to GM-CSF. However, structural analysis of the CDR using molecular modeling revealed potential mimickry of the binding region of GM-CSF. Monfardini et al. demonstrated that the CDR mimickry of the three-dimensional structure of the antigen could be used to design a model of the binding region of GM-CSF, and that this model could potentially be applied to drug design.

In other words, antibodies can be raised to the topology of a small molecule (or any ligand) with a desirable binding affinity to a given target protein. The CDR regions of these antibodies structurally encode an imprint of the ligand topology. This is assumed to be maintained even in the absence of the ligand. It follows that the encoded topology in the CDR domain should represent a similar structural and electrostatic environment as the ligand binding site on the target protein (usually an enzyme or receptor).

Similarly, one could prepare an antibody to this antibody (also known as an anti-idiotype antibody). It would be expected in such an anti-idiotypic antibody that the CDR-encoded structure would resemble the surface topology of the initial ligand.

The CDRs of anti-idiotype antibodies may be sequenced and used to construct model peptide sequences, as described in U.S. Pat. No. 5,637,677 (Greene, et al.), incorporated by reference herein. The anti-idiotype antibodies may also be used for the design and synthesis of peptide or non-peptide compounds (mimetics) which would be useful for the same diagnostic and therapeutic applications as the antibodies, as for example in Saragovi et al., (1991) Science 253, 792-795, and U.S. Pat. No. 5,817,756, herein incorporated by reference.

### Immunization

Next, an animal is immunized with the monoclonal antibodies to generate anti-idiotype monoclonal antibodies reactive against the antigen binding region of the immunizing monoclonal antibodies. In an alternative embodiment, the immunization is performed in vitro, for example using splenocytes harvested from an organism. If a specific antibody is used as an immunogen, the antibody itself will result in an immune response. Anti-antibodies are produced that are specific for the unique epitopes (idiotopes) of the variable domains of the injected antibodies; these epitopes are known collectively as the idiotype of the primary antibody and the secondary (anti-) antibodies which bind to these epitopes are known as anti-idiotypic antibodies. Other secondary antibodies will be specific for the epitopes of the constant domains of the injected antibodies and hence are known as anti-isotypic antibodies.

In a preferred embodiment of the present invention, anti-idiotypic antibodies are prepared using the antibodies prepared as described above. The antibodies produced in response to immunization with the primary antibodies (the antibodies defining the structure of the compound that generically defines the surface conformation and surface charge density) are screened for reactivity against the primary antibodies. Further screening for reactivity against nonspecific antibodies is performed in order to eliminate those antibodies with anti-isotype reactivity. Only anti-idiotypic antibodies which indicate a specific reactivity with the antigen binding region of the primary antibody are selected for further study

### Three-Dimensional Structure

The three-dimensional structure of the antigen binding region of the anti-idiotypic antibodies is determined.

There are several methods used routinely to determine the three-dimensional structure of proteins; in this case the peptidic CDR segments of these anti-idiotype antibodies, The best, most reliable method is simply to solve the crystal structure of the antibody of interest. With that data in hand, methods are known widely and commonly for using those atomic coordinates to construct three dimensional models on computers. Prior to use in drug design, models such as these are first “energy minimized” using either first or second derivative function minimization techniques such as the steepest descents, conjugate gradients, or Newton-Raphson approaches. Briefly, the energetics of a molecule are represented by a force field such as the following, where the total energy of a given set of coordinates, atom types, and their connectivities is a function of several individual terms.

E_{TOT}=ΣE_{Bond}+ΣE_{angle} ΣE+ΣE_{vdw}+ΣEₑₗₛₜₐₜ+. . .

In an iterative fashion, energy minimization techniques perturb atomic positions, then re-evaluate the total energy. The goal is to find the lowest local energy minima, when one starts from crystallographically determined starting coordinates. In a crystalline state, atoms can pack too tightly, driven by forces associated with the crystalline lattice. These strong forces are thought to exist only in solid states, not solution states where biological activity would be more relevant.

Hence, energy minimizing a crystal structure would be a good technique to cause an appropriate relaxation of these forces. Moreover, this “relaxed” state is generally considered to be of more biological relevance. Programs typically used for this include Quanta, CIIARMm, Insight and Discover.

Frequently, it is not possible to obtain a crystal structure, however, there might be “related” crystallography available. In other words, an antibody of the same class might have been solved by crystallography, the only difference being the CDR peptide segments. In this case, a technique known as homology modeling might be used to create a best guess of the three dimensional structure of the desired antibody. Conceptually, the non-CDR part of the antibody is assumed to adopt the same structure, regardless of the CDR domain. The CDR regions are constructed one at a time by taking the primary amino acid sequences, and searching for their occurrence in databases of solved crystallographic structures of many proteins. The most used database is the one at Brookhaven National Laboratories, available via the internet. Matching coordinates are ultimately substituted into the model. As an alternative, de novo predictions can be made regarding the structure of these CDR domains, but these are generally less reliable. Molecular Dynamics and/or energy minimization are used routinely in this homology modeling process as well.

Given the primary amino acid sequence of a CDR, one can use available computational tools lit (including energy minimization and molecular dynamics) to deduce a Three dimensional structure of the binding site. This structure will be energetically described by terms that account for electrostatic charges, Van der Waals radii, bond lengths, etc. Such a representation can be used to search through electronic databases of putative ligand by “docking” each to the site in a variety of orientations, then assigning a score based on goodness of fit (complementarity). Compounds with a high score would be candidates for a real experimental assay. There are many programs available for this type of ligand search including DOCK, RECEPTOR, LEAPFROG. In addition, visual inspection of the site models can be used by medicinal chemists to design complementary structures de novo.

In a preferred embodiment, the characterization of the CDR structures of the antibodies having affinity for a desired compound that generically defines the surface conformation and surface charge density of the small organic molecule, inorganic molecule, biological molecule or molecule class is used to determine the optimum molecular characteristics of the receptor or binding site for a compound or compound class.

Similarly, characterization of the binding CDRs of the anti-idiotypic antibodies provides a method for determining the optimum molecular characteristics of the desired compound that generically defines the surface conformation and surface charge density of the small organic molecule, inorganic molecule,.biological molecule or molecule class, because the structure of the antigen binding region of the anti-idiotypic antibody is a mirror image of the antigen binding site of the idiotypic antibody, and acts as a mimic of the original antigen/hapten structure.

Anti-idiotypic antibodies possessing the greatest affinity and specificity for the antigen binding region of the primary antibody are chosen for further studies designed to elucidate the structure adopted by the CDR of the anti-idiotypic antibodies. Several methods are available. For example, the antibodies can be subjected to x-ray crystallography in order to ascertain the structure of the CDR. The three-dimensional electron density map of the surface contours of the anti-idiotypic antibody CDR provides valuable information concerning the shape and contour needed for the design of additional desired compounds.

In addition, the conformation of the CDR peptide itself can be studied using x-ray crystallography in conjunction with molecular modeling and spectroscopic techniques such as NMR, FTIR or CD. The spectroscopic analysis provides information about conformational constraints for potential peptide structures, and can be used to enhance and refine the molecular modeling of the peptide. The information from modeling and spectroscopic studies, alone, or in combination with x-ray diffraction data, provides meaningful three-dimensional parameters which provide guidance toward the design of additional useful compounds.

In further embodiments, an anti-anti-idiotypic antibody can be generated against the anti-idiotypic antibody, to determine whether the anti-idiotypic antibody is in fact the image of the generic molecule. The anti-anti-idiotypic antibody will compete with the primary antibody for binding to the compound that generically defines the surface conformation and surface charge density if it is specific for the antigen binding region of the anti-idiotypic antibody. Alternatively, competitive binding experiments can be performed that will establish that the anti-idiotypic antibody binds to the same site as the compound that generically defines the surface conformation and surface charge density, its analogs or any additional species known to possess inhibitory activity against the desired compound.

Examples of small organic molecules, inorganic molecules, biological molecules or molecule classes suitable for use in conjunction with the present invention include many drugs and active agents known to those skilled in the art. For example, the invention can be used to screen for many active agents, including but not limited to ligands for the following binding sites: galanin, interleukin-1, interleukin-4, interleukin-5, interleukin-11, chemokines, cholecystokinin, leptin, glucose transporters, proteases, glutamate receptors, neuropeptideY, neurokinin-1, neurokinin-2, neurokinin-3, bombesin, cannabinoid, gastrin, corticotropin releasing hormone, dopamine, endothelin, orphanin, calcium channels, sodium channels, potassium channels, transcription factors, bacterial enzymes, viral enzymes. Thus, while the invention is particularly suited to screening for medicinal purposes, it is contemplated that the invention will be used for the design and screening is of compounds useful in agriculture and chemical manufacturing.

### PDE IV Inhibitors

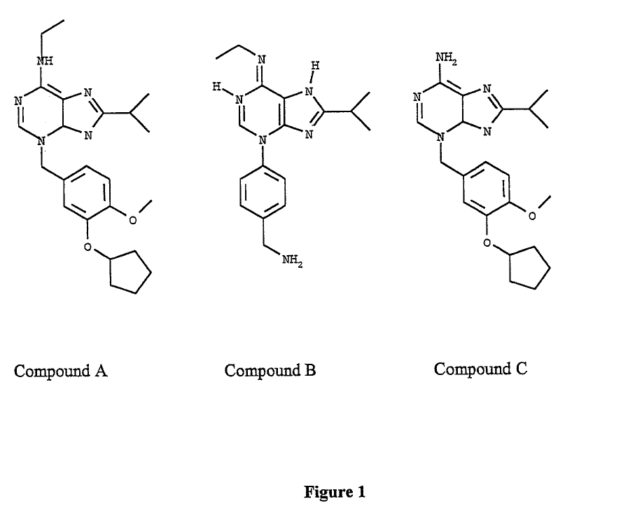

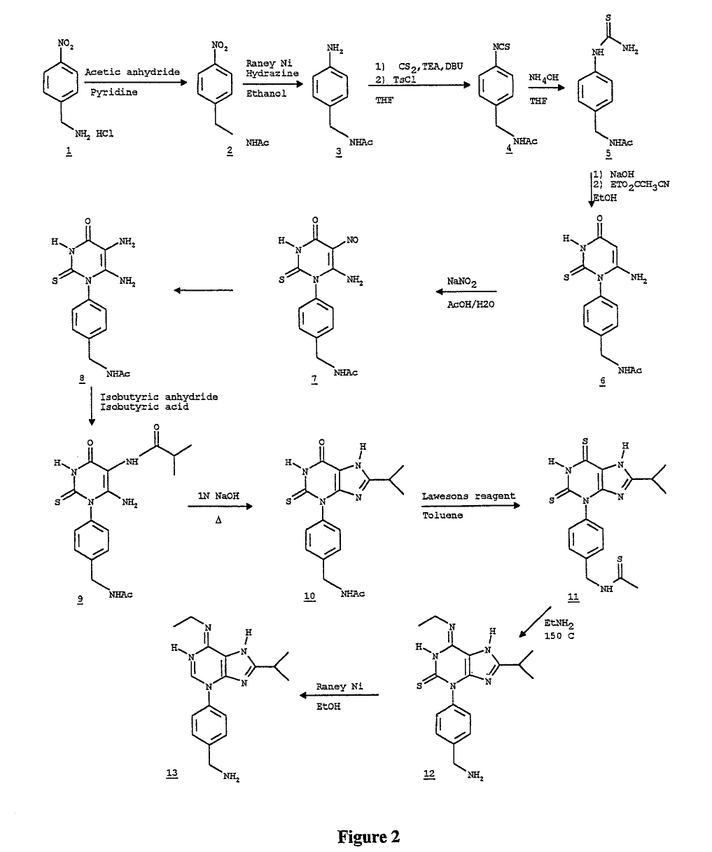

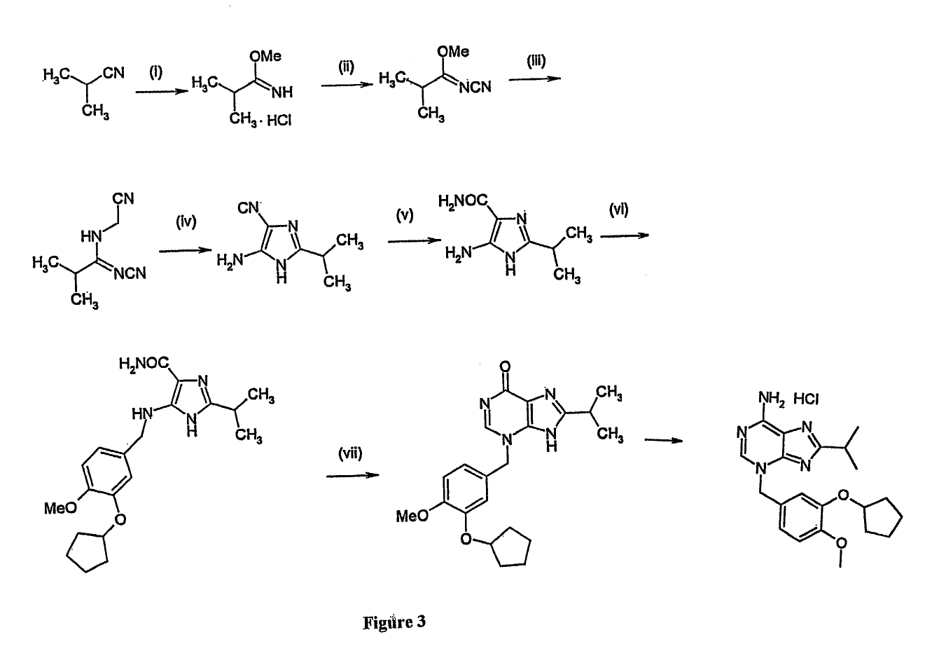

In one preferred embodiment of the invention, the small organic molecule has selective Phosphodiesterase IV (“PDEIV”) inhibitory activity. For example, the compound that generically defines the surface conformation and surface charge density of a prototype PDEIV inhibitor is 3-(3-cyclopentyloxy-4-methoxybenzyl)-6-ethylamino-8-isopropyl-purine, hereinafter referred to as “Compound A”. Compound A is chosen as a compound that generically defines the surface conformation and surface charge density of the small organic molecule or molecule class, and analogs are designed which may be covalently linked to a carrier molecule. The structures of Compound A and two analogs of this class of PDEIV inhibitor, 6-ethylamino-8-isopropyl-3-(p-aminomethylphenyl)-purine (“Compound B”), and 3-(3-cyclopentyloxy-4-methoxybenzyl)-8-isopropyl-3H-purine (“Compound C”) are shown in FIG. 1. The analogs can be prepared in various manners known to one skilled in the art. For example, Compound B, is prepared using the synthetic scheme shown in FIG. 2 and Compound C using the synthetic scheme shown in FIG. 3.

As seen in FIG. 1, the analogs of Compound A have free amino groups for coupling to KLH (i.e. they are haptenized). In particular, the free amino functional group of Compound B, a part of the 3-p-aminomethylphenyl substituent, is designed to react with succinylated KLH to form a covalent linkage, using carbodiimide. The adenine moiety of Compound B is exposed to the solution as a result of its haptenization, and monoclonal antibodies generated by immunization with this conjugate are selective for the 6-ethylamino-8-isopropyl substituted adenine moiety. The haptenization results in the generation of monoclonal antibodies having specificity for the solvent exposed portion of Compound A. The sterically hindered portion of the analog does not elicit a specific antibody reaction because it is not accessible to the antibodies. Thus, antibodies specific for Compound B define the substituted adenine moiety of Compound A which binds to the PDEIV binding site.

Likewise, the analog Compound C has a free amino functional group at the adenine nucleus. Antibodies directed against the Compound C hapten exhibit specificity for the 3-cyclopentyloxy-4-methoxybenzyl substituent, because this moiety is exposed to the solution. Thus antibodies which bind Compound C with specificity define the 3-cyclopentyloxy-4-methoxybenzyl moiety of Compound A, which binds to the PDEIV binding site.

Further monoclonal antibodies having specificity for further aspects of Compound A can be generated in the same fashion, such that monoclonal antibodies to a portion, or the entire surface of Compound A are created. For example, additional analogs that may be attached to a carrier molecule are rolipram, 3-(3-cyclopentyloxy-4-methoxybenzyl)-6-ethylamino-8-isopropyl-purine or Benzamide, 3-(cyclopentyloxy)-N-(3,5-dichloro-4-pyridinyl)-4-methoxy-(9CI) (known as “Piclamilast” or “RP73401”), wherein the analogs are attached via an ether linkage from the 4-O to the carrier molecule. See, Ashton, Michael J.; Cook, David C.; Fenton, Garry; Karlsson, Jan-Anders;Palfreyman, Malcolm N.; Raebum, David; Ratcliffe, Andrew J.; Souness, John E.; Thurairatnam, Suga; Vicker, Nigel, Selective Type IVPhosphodiesterase Inhibitors as Antiasthmatic Agents. The Syntheses and Biological Activities of 3-(Cyclopentyloxy)-4 methoxybenzamides and Analogs, J. Med. Chem.37(11), 1696-703, (1994); and Raeburn, David; Underwood, Stephen L.; Lewis, Susan A.; Woodman, Valerie R.; Battram, Cliff H.; Tomnkinson, Adrian; Sharma, Steven; Jordan, Roy; Souness, John E.; et al, Anti-inflammatory and bronchodilator properties of RP 73401, a novel and selective phosphodiesterase type IV inhibitor, Br. J. Pharmacol., 113(4), 1423-31, (1994), hereby incorporated by reference.

Yet another analog that may be attached to a carrier molecule is cyclohexanecarboxylic acid, -4-cyano-4-[3-(cyclopentyloxy)-4-methoxyphenyl]-, cis-(9CI) known as “Ariflo” or “SB 207499”), See, Barnette, Mary S.; Christensen, Siegfried B.; Essayan, David M.; Grous, Marilyn; Prabhakar, Uma; Rush, Julia A.; Kagey-Sobotka, Anne; Torphy, Theodore J., SB 207499 (Ariflo), a potent and selective second-generation phosphodiesterase 4 inhibitor: in vitro anti-inflammatory actions, J. Pharmacol. Exp. Ther., (1998), 284(1), 420-426; and Christensen, Siegfried B.; Guider, Aimee; Forster, Cornelia J.; Gleason, John G.; Bender, Paul E.; Karpinski, Joseph M.; DeWolf, Walter E. Jr.; Barnette, Mary S.; Underwood, David C.; Griswold, Don E.; Cieslinski, Lenora B.; Burman, Miriam; Bochnowicz, Steven; Osborne, Ruth R.; Manning, Carol D.; Grous, Marilyn; Hillegas, Leonard M.; Bartus, Joan O'Leary; Ryan, M. Dominic; Eggleston, Drake S.; Haltiwanger, R. Curtis; Torphy, Theodore J., 1,4-Cyclohexanecarboxylates: Potent and Selective Inhibitors of Phosophodiesterase 4 for the Treatment of Asthma, J. Med. Chem., 41(6), 821-835, (1998) incorporated by reference hereto.

A further example of an additional analog that may be attached to a carrier molecule is pyridine,4-[(2R)-2-[3-(cyclopentyloxy)-4-methoxyphenyl]-2-phenylethyl]-(9CI) (known as “CDP 840”) See Hughes, B.; Howat, D.; Lisle, H.; Holbrook, M.; James, T.; Gozzard, N.; Blease, K.; Hughes, P.; Kingaby, R.; et al., The inhibition of antigen-induced eosinophiliaand bronchoconstriction by CDP840, a novel stereo-selective inhibitor of phosphodiesterase type 4, Br. J. Pharmacol., 118(5), 1183-1191 (1996), hereby incorporated by reference.

An additional compound that generically defines the surface conformation and surface charge density of the small organic molecule or molecule class is 3,5-di-t-butyl-4-hydroxy-benzyl-5-chloro-2-(2-pyridyl)-ethynyl-benzoxazole, hereinafter referred to as “Compound D”, which can be utilized as the compound that generically defines the surface conformation and surface charge density of the small organic molecule or molecule class of PDEIV inhibitors. For example, Compound D and its analogs can be attached to a carrier molecule via the 4-hydroxy using an ether linkage.

The monoclonal antibodies generated from these analogs define the surface conformation of these PDEIV inhibitors. The relative affinity of the antibodies for different PDEIV inhibitors provides a method of defining the genus of chemical compounds to which each tested compound belongs. As discussed above, antibodies able to discriminate between compounds can be used to generate structure-activity relationships for design of additional compounds exhibiting PDEIV inhibitor activity. These antibodies can also be used for quantitative analysis of binding affinity and theoretical modeling of the PDEIV inhibitor or PDEIV inhibitor candidate design. The antibodies may also be used to model compounds which selectively bind to additional receptor or enzyme subtypes, such as the PDEIII binding site, or to define the conformation of PDEIU inhibitors.

Preferably, the monoclonal antibodies are used to mimic the PDEIV binding site, as well as to define the surface conformation of the hapten or compound that generically defines the surface conformation and surface charge density of a PDEIV inhibitor. For example, competitive binding experiments are used to characterize the antibody selectivity and specificity between the Compound B-KLH or Compound C-KLH conjugates and additional known PDEIV inhibitors or substrates and the ability of the antibodies to discriminate between these species is determined.

IC₅₀ is a measure of the ability of a compound to inhibit the response of a receptor to a known agonist or to inhibit the catalytic activity of an enzyme. In other words, IC₅₀ can serve as a measure of the relative affinities between the inhibiting compound(s) and the agonist or inhibitor for the receptor binding site (e.g., enzyme active site), respectively. The binding affinities of the monoclonal antibodies for the inhibiting compound(s) are determined by means of competitive binding assays for: 1) the hapten conjugates; 2) the compound that generically defines the surface conformation and surface charge density; and/or 3) additional compounds known to bind to the receptor binding site (e.g., enzyme active site). The binding affinities of the monoclonal antibodies can be compared with the measured IC₅₀ values in order to select those which sufficiently match the receptor binding site (e.g., enzyme active site) binding characteristics.

Using comparative antibody affinities for test compounds and measured IC₅₀ values, the monoclonal antibodies can be used to model the PDEIV binding site itself, and to predict structures of additional PDEIV inhibitors. For example the IC₅₀ for Compound A is 410 nM, while the IC₅₀ for Compound C is 800 nM and for rolipram the IC₅₀ is 3000nM. Additional examples of PDEIV inhibiting compounds can be included in this database, and are exemplified in co-pending U.S. patent application Ser. No. 08/647,419, filed May 9, 1996, U.S. patent application Ser. No. 08/782,502, filed Jan. 10, 1997, U.S. patent application Ser. No. 08/875,487, filed Jul. 8, 1997, U.S. patent application Ser. No. 08/659,767, filed Jun. 6, 1996, U.S. patent application Ser. No. 08/714,581, filed Sep. 16, 1996, U.S. patent application Ser. No. 08/833,893 filed Apr. 10, 1997, U.S. patent application Ser. No. 08/860,680, filed Jun. 11, 1997, U.S. patent application Ser. No. 08/860,674, filed Jun. 11, 1997, U.S. patent application Ser. No. 09/151,949, filed Sep. 11, 1998, and U.S. patent application Ser. No. 08/963,054, filed Nov. 11, 1997, the disclosures of which are all incorporated by reference herein in their entirety.

Structure-activity relationships for the design of additional PDEIV inhibitor structures can be generated using IC₅₀ data. The binding affinity of the monoclonal antibodies for PDEIV inhibitors can be used in a similar way to generate structure-activity relationships for PDEIV inhibitors. The binding affinity of the antibodies can also be used for theoretical modeling of the PDEIV binding site or PDEIV inhibitor candidate design.

Once the ability of the monoclonal antibodies to discriminate between PDEIV inhibiting species and to mimic the PDEIV binding site is established, the antibodies are used to screen unknown compounds for PDEIV inhibiting compounds. A panel of antibodies is created from those monoclonal antibodies having a binding affinity over 10⁸ for use in determinations of the surface structure of the Compound A molecule and in tests for screening for molecules having PDEIV inhibition activity. Preferably, the panel is comprised of at least two antibodies and more preferably the panel is comprised of between 2-3 antibodies.

New compounds are screened for PDEIV inhibitor activity using the antibody panel. This may be accomplished by various methods known in the art, as discussed above. In one preferred example, the antibodies are coupled to an affinity matrix and used to capture components from the solution which exhibit a similar surface conformation, solvation and surface charge density to PDEIV inhibitors. In another preferred example, a screening method is used to determine the ability of solutions with unknown components to competitively inhibit the binding of the Compound A analog-KLH conjugate to the antibodies. Both of the previous screening methods are equally applicable to known compounds, either in mixtures or in pure form.

### DETAILED DESCRIPTION OF THE PREFERRED EMBODIMENTS

The following examples illustrate various aspects of the present invention. They are not to be construed to limit the claims in any manner whatsoever.

### EXAMPLE 1

In Example 1, a compound having a generic binding site to Compound A is subjected to high-throughput screening as follows:

First Compound B is synthesized according to the process as set forth FIG. 2 and explained below.

a) Synthesis of p-nitrobenzylacetamide (FIG. 2, product 2): A 500 ml 3-neck flask fitted with a reflux condenser and a thermometer was charged with 150 ml pyridine. The reaction was cooled to 0° C. in an ice bath with magnetic stirring. The 4-nitrobenzylamine hydrochloride (1) (25 g, 0.133 mol) was added in two 12.5 g portions each followed by acetic anhydride (8.1 g, 0.08 mol each addition). The temperature rose to ca. 10° C. and was allowed to cool back down to 0° C. before the next addition. After the second addition a precipitation began to form. The reaction was stirred for 30 min more at 0° C. and then removed from the ice bath and allowed to warm to room temperature over another 30 minutes. The reaction was diluted with 200 ml of water and extracted 2×100 ml of dichloromethane. The organic layers were combined, dried with sodium sulfate, filtered through 1-PS paper, and stripped on a rotary evaporator to yield an orange oil. Toluene (2×100 ml) was added to the flask and then stripped to remove excess pyridine. The product was obtained as an off-white solid (22.1 g 86% yield, TLC Rf 0.4 EtOAc).

b) Synthesis of p-aininobenzylacetamide (FIG. 2, product 3): A 3-neck round bottomed flask fitted with a reflux condenser, a thermometer, and mechanical stirrer was charges with p-nitrobenzylacetamide (22.1 g, 0.114 mol) and ethanol (200 ml). The reaction was warmed on a steam bath to give a light yellow solution. The Raney nickel was added with an additional 50 ml of ethanol. The reaction was warmed to 70° C. and hydrazine (91.4 g 0.23 mol) was added dropwise over a period of 30 minutes. The reaction was monitored by TLC 10% MeOH/CH₂Cl₂ product R_{f}=0.25. The reaction was filtered through celite, stripped on a rotary evaporator, and then dried on a high vacuum line yielding the product 3 as a white solid (17 g, 90% yield).

c) Synthesis of p-isothiocyanatobenzylacetamide (FIG. 2, product 4): A 500 ml 3-neck round bottomed flask fitted with a reflux condenser and a mechanical stirrer was charged with p-25 aminobenzylacetamide (product 3) (15.5 g 0.095 mol), tetrahydrofuran (100 ml), carbon disulfide (206. G, 0.27 mol), triethylamine (36.6 g, 0.36 mol), and DBU (20.6 g, 0.136 mol). The addition of triethylamine caused a yellow precipitate to form and the addition of the DBU caused an orange oil to form. The reaction was then cooled in a water bath and stirred at room temperature to 1.5 hours. TLC analysis (10% MeOH.CH2Cl2) showed primarily one spot (Rf=0.2) with some faster moving components. After a total of 4 hours of reaction the TLC was virtually unchanged. The p-toluenesulfonyl chloride was then dissolved in 50 ml of dichloromethane and added dropwise to the rapidly stirring reaction which was cooled in a water bath. The reaction was stirred for 30 minutes after the addition of the p-toluenesulfonylchloride at which time the TLC showed no intermediate (R_{f}=0.22) and two other spots R_{f}=0.25 and one at R_{f}=0.5 corresponding to product. The reaction was then poured into 500 ml of water and the layers were separated. The aqueous phase was then extracted with 100 ml of dichloromethane. The dichloromethane layers were combined and washed with 3×100 ml 1N NaOH, 2×100 ml water, dried with sodium sulfate, filtered, and concentrated on a rotary evaporator until a precipitate formed. The precipitate was filtered and a second crop was harvested from the concentrated mother liquors. A total of 13.0 g of product was isolated (67% yield).

d) Synthesis of p-thioureidobenzylacetamide (FIG. 2, product 5). A slurry of p-isothiocyanatobenzylacetamide (product 4) (13 g, 0.063 mol) in 75 ml of THF was rapidly stirred as 30% aqueous ammonia (21 ml, 0.32 mol) was added over a period of 20 minutes. After an additional 10 minutes the reaction was homogeneous and the TLC showed one spot (R_{f}=0.2, 10% MeOH/CH₂Cl₂). The reaction was diluted with 100 ml of dichloromethane and filtered. The filter cake was dried in a vacuum oven yielding 13.0 g of product in 93% yield.

e) Synthesis of 4-amino-3-(p-acetarnidomethylphenyl)-2-thiouracil (FIG. 2, product 6). Ethanol (150 ml) was dried over 3 Angstrom molecular sieves. The ethanol and p-thiouridobensylamide (product 5) were combined in a 500 ml 3-neck flask fitted with a mechanical stirrer, and reflux condenser. To the rapidly stirring slurry 11.3 ml of 1 M sodium ethoxide in ethanol was slowly added. The reaction became homogeneous and the ethyl cyanoacetate (9.9 g, 0.087 mol) was added. The reaction was then heated to reflux in an oil bath. The progress of the reaction was monitored by TLC (10% MeOH.Ch2Cl2) and after 15 hours 5 ml more of ethyl cyanoacetate was added. The TLC remained little changed after 5 hours so 2 ml more of 1 M sodium ethoxide was added. The reaction continued for another 2 hours. After this time 10 ml more ethyl cyanoacetate was added and 80 ml of ethanol was distilled out of the reaction. The reaction was continued another 15 hours and the TLC remained unchanged. The temperature was raised to 120° C and the reaction continued for an additional 8 hours. After this time the reaction was cooled to room temperature and allowed to stir an additional 17 hours. The reaction was then diluted with 200 ml of ether and 50 ml of dry ethanol. The resulting precipitate was filtered and then triturated with 100 ml of ethanol. The tan solid was slurried with methanol then acidified with 2.4 g of acetic acid. The solid was filtered and washed with methanol to give 11 g of (product 6) as an off-white solid (65% yield).

f) Synthesis of 4-amino-5-nitroso-3-(p-acetamideomethylphenyl)-2-thiouracil (FIG. 2, product 7). A 250 ml 3-neck round bottom flask fitted with a mechanical stirrer, reflux condenser, and pressure equalizing addition funnel was charged with 4-amino-3-(p-methylacetamidophenyl)-2-thiouracil (product 6) (10 g, 35 mmol), acetic acid (50 ml), and water (50 ml). The reaction was heated on a steam bath to 75 ° C., during which time the reaction became homogeneous and straw colored. The addition funnel was charged with a solution of sodium nitrate (2.62 g, 38 mmol) dissolved in 10 of water. This solution was then added to the rapidly stirring reaction maintaining the temperature between 70-80° C. The temperature was maintained for another 15 minutes and then the reaction was cooled in an ice water bath. When cold the reaction was filtered and the crystals were washed with a small amount of water. The product (product 7) isolated as bright blue crystals (10.5 g, 95% yield).

g) Synthesis of 4,5-diamino-3-(p-acetamidomethylphenyl)-2-thiouracil (FIG. 2, product 8). The 4-amino-5-nitroso-3-(p-methylacetamidophenyl)-2-thiouracil (product 7) was added to 35 ml of 1N NaOH in a 100 ml round bottom flask. The resulting red gooey reaction was magnetically stirred as the sodium dithionite (9.2 g, 66 nuol) was added with the aid of 5 ml of 1N NaOH. The reaction was stirred for 45 minutes longer at which time no red material remained. The reaction was filtered and washed with 30 ml of water. The solid was then triturated with 50 ml of ether then 20 ml of ethanol was added and the solid was triturated further, 50 ml more of ether was added and the solid was filtered. The product was air dried to give 5.5 g of off white powder (82% yield).

h) Synthesis of 4-amino-5-isobutyramide-3-(p-acetamidomethylphenyl)-2-thiouracil (FIG. 2, product 9). A 50 ml 3-neck flask fitted with 2 glass stoppers and a reflux condenser was charged with 30 ml of isobutyric acid, isobutyric anhydride (3.42 g, 22 mmol), and 4,5-diamino-3-(p-methylacetamidophenyl)-2-thiouracil (product 8) (5.5 g, 18 mmol). The reaction was magnetically stirred while heated on a steam bath. After 30 minutes the reaction turned into a solid mass. The reaction was continued for another 45 minutes and then cooled to room temperature at which time it was filtered. The filter cake was pressed dry, washed with 100 ml of ether and air dried for several hours. The material was carried on without further purification.

i) Synthesis of 8-isopropyl-3-(p-acetamidomethlyphenyl)-2-thiopurine (FIG. 2, product 10). A 250 ml 3-neck flask fitted with 2 glass stoppers and a reflux condenser was charged with 100 ml of 1 N NaOH and the solid from the previous reaction. The solution was magnetically stirred and heated to a vigorous reflux in an oil bath. The reaction was monitored by TLC and after 1 hour no starting material (Rf 0.23, 10% MeOH.CH2Cl2) was evident and only one spot for product was detectable (Rf 0.62). The reaction was cooled to room temperature and neutralized with 6 g of acetic acid. The resulting mixture was extremely difficult to filter and took overnight to completely filter. The solid was recrystallized from methanol to give 4 g of product (62% yield from product 8).

j) Synthesis of 8-isopropyl-3-(p-thioacetamidomethylphenyl)-2,6-dithiopurine (FIG. 2, product 11). A 3-neck Morton flask fitted with 2 glass stoppers and a reflux condenser was charged with 8-isopropyl-3-(p-methylacetamidophenyl)-2-thiopurine (10) (2.9 g, 8.1 mmol), Laweson's reagent (7.22 g, 17.9 mmol), and 70 ml of toluene. The reaction was magnetically stirred and heated to reflux in an oil bath. The reaction was monitored by TLC (10% MeOH/CH2C12, product Rf 0.6) and was over in 3 hours. The reaction was cooled in an ice bath and filtered. The precipitate was triturated with methanol and filtered. The filtrate was stripped yielding a residue which was primarily product by TLC. The residue was submitted to flash chromatography on 50 g of flash silica gel with 20 g of sodium sulfate on top. The column was eluted with hexane/dichloromethane with 1% methanol. The product came off in 40% dichloromethane with 1% methanol. The column fractions containing one spot by TLC were combined and stripped yielding 2 g of pure product (63% yield).

k) Synthesis of 6-ethylan-ino-8-isopropyl-3-(p-aminomethylphenyl)-2-thio-purine (FIG. 2, product 12). The 8-isopropyl-3-(p-thioacetamidomethylphenyl)-2,6-dithiopurine (product 11) (2g, 5.1 mmol) was dissolved in 15 ml of 70% aqueous ethylamine. The solution was heated to 150 C in a bomb for 17 hours. The contents of the bomb were stripped and the residue was dissolved in hot methanol and treated with Norit A charcoal. The charcoal was filtered and 20 g of the sodium sulfate was added to the methanol solution. The methanol was then stripped from the slurry leaving the product adsorbed onto the sodium sulfate. The sodium sulfate was then added on top of 50 g flash silica gel column. The column was eluted with hexane/dichloromethane/methanol/triethylamine with the product coming off the column in 10% methanol/89% methylene chloride/1% triethylamine. The column fractions containing one spot by TLC for the product (Rf=0.8, 5 ml triethylamine/35 ml methanoV65 ml dichloromethane) were combined and stripped, yielding 1.1 g of product 12 in 63% yield.

l) Synthesis of 6-ethylamino-8-isopropyl-3-(p-aminomethlyphenyl)-purine (FIG. 2, product 13). A 50 ml round bottom flask was charged with 6-ethylamino-8-isopropyl-3-(p-aminomethylphenyl)-2-thiopurine (product 12) (900 mg, 2.6 mmol) and 5 ml of 1.5 N NaOH in ethanol. To this rapidly stirring solution Raney Ni (4 g) was added with the aid of 5 ml of 1N NaOH in ethanol. The reaction was stirred at room temperature for 30 minutes. After this time the TLC shows the presence of two spots Rf=0.75 and 0.82 (5% triethylamine/20% methanol/75% dichloromethane). The reaction was then filtered through celite and the celite was washed with 3×50 ml of ethanol. The filtrates were combined and concentrated on the rotary evaporator. The reaction was acidified with 1:10 HCL and then the pH was brought to 7-8 with saturated sodium bicarbonate. The reaction was extracted with 4×25 ml of chloroform. The chloroform layers were combined and filtered through 1-PS paper and stripped to yield 350 mg of residue. The residue was then submitted to a 2 mm silica gel chromatatron. The chromatatron was eluted with 70% dichloromethane/28% hexane/1% methano/11% triethylamine. The chromatatron yielded 380 mg (46% yield) of material that did not crystallize. This material was dissolved in 10 ml ethanol and combined with a solution of 143 mg fumaric acid dissolved in 10 ml hot ethanol. As the flask was allowed to cool to room temperature, crystals began to form. The flask was stored in the freezer overnight and the resulting crystals were filtered to give 450 mg of final product (product 14).

Next, Compound B (an analog of Compound A) was conjugated to succinylated keyhole limpet hemocyanin (sKLH). After the coupling reaction, samples were analyzed by measuring UV absorbance. Extinction coefficients were calculated for Compound B, KLH, and sKLH. By comparing the absorbance spectra of these samples to coupled-reaction samples, it was determined that Compound B had been successfully coupled to sKLH at a ratio of 25 molecules of Compound B to 1of sKLH.

All coupled-reaction samples have a concentration of ˜2 mg/ml which is appropriate for injection into animals for production of monoclonal antibodies. The coupling procedure was accomplished as follows:

1: sKLH was dissolved and diluted to a concentration of 2 mg/ml in borate buffered saline (BBS).

2: Compound B was added to the tube to a final concentration of ˜1 mg/ml and 1-ethyl-3-(3-dimethylaminopropyl) carbodiimide methiodide to a final concentration of 0.4 mg/ml.

3: The reaction was allowed to incubate at room temperature while being vortexed for 2 hours at a setting of 2.

4: The sample was transferred to dialysis tubing (MW cutoff 8,000) and dialyze against BBS.

5: The sample was collected after dialysis and analyze part of it by UV absorbance.

Next, Compound C (an analog of Compound A) was conjugated to succinylated keyhole limpet hemocyanin (sKLH). After the coupling reaction, samples were analyzed by measuring UV absorbance. Extinction coefficients were calculated for Compound C, KLH, and sKLH. By comparing the absorbance spectra of these samples to coupled-reaction samples, it was determined that Compound C was coupled to sKLH at a ratio of 39 to 1 using methanol as solvent (use of solvent will be explained below), 23 to 1 using dimethyl sulfoxide, 17 to 1 using dioxane, and 13 to 1 using reagent grade alcohol (95% ethanol and 5% isopropanol).

1: 6 mg Compound C was dissolved in 2 ml methanol in 50 ml centrifuge tube (methanol was used because Compound C is insoluble in aqueous solution).

2: 1 ml of 5× BBS was added to the tube while vortexing at a setting of 2.

3: sKLH was dissolved to a concentration of 5 mg/ml in 2 ml of water (as per instructions from Sigma product sheet) and then added these 2 ml while vortexing at a setting of 2.

4: 1-ethyl-3-(3-dimethylaminopropyl) carbodiimide methiodide was added to a final concentration of 0.4 mg/ml-2 mg in 5 ml.

5: The reaction was allowed to incubate at room temperature while being vortexed for 2 hours at a setting of 2.

4: The sample was transferred to dialysis tubing and dialyzed against BBS.

5: The sample was collected after dialysis and analyze by UV absorbance.

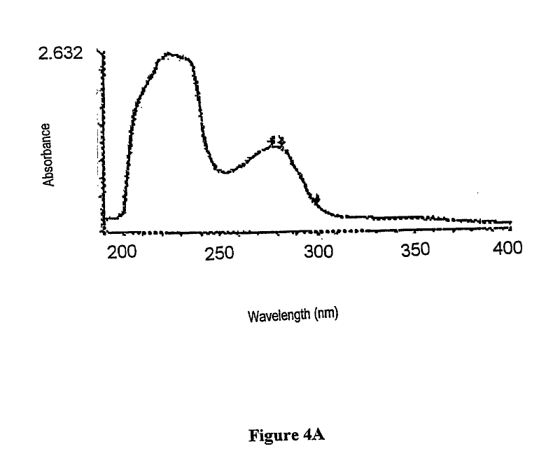

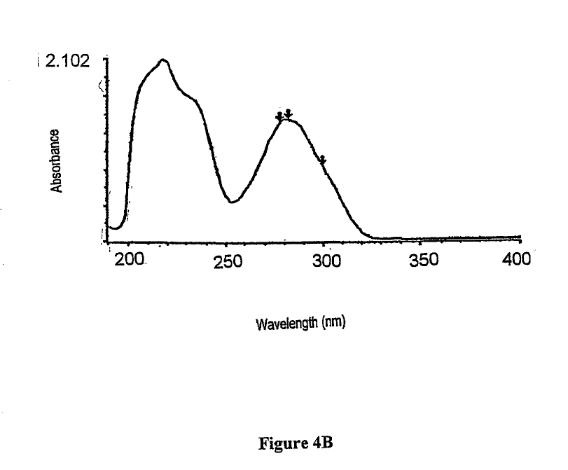

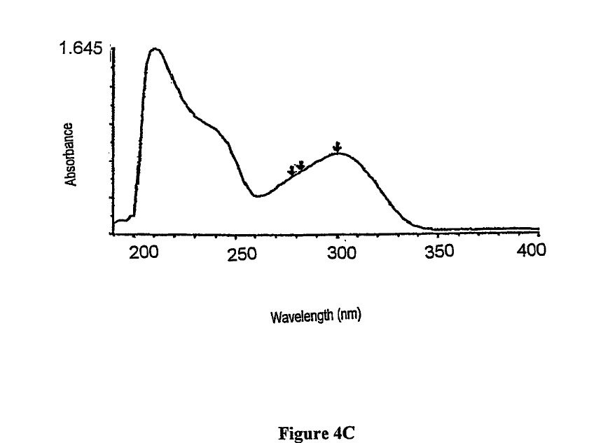

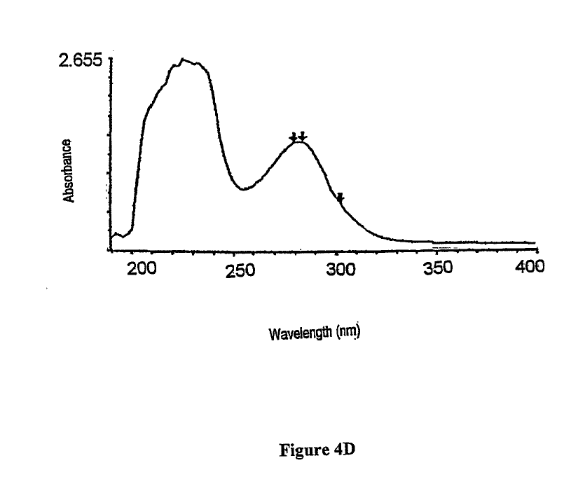

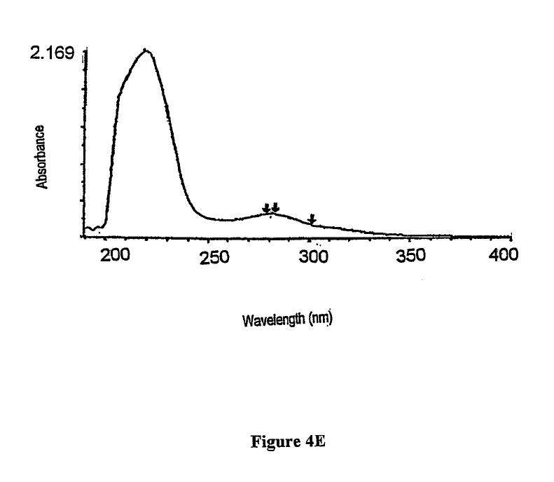

Coupling efficiencies were analyzed by measuring UV absorbance spectra of sKLH (FIG. 4A), Compound C (FIG. 4B), Compound B (FIG. 4C), Compound C coupled to sKLH (FIG. 4D), and Compound B coupled to sKLH (FIG. 4E).

TABLE 1
Absorbance Values at 300, 282 at 278 nm

| Analyte | 300 nm | 282 nm | 278 nm |
|---|---|---|---|
| Succinylated KLH | 0.3772 | 1.2415 | 1.2543 |
| Compound C (0.025 mg/ml) | 0.8731 | 1.4202 | 1.3851 |
| Compound B (0.02 mg/ml) | 0.7129 | 1.5460 | 0.5021 |
| Compound C-KLH (1.0 mg/ml) | 0.6419 | 1.5031 | 1.4953 |
| Compound B-KLH (1.0 mg/ml) | 0.1488 | 0.2752 | 0.2706 |

Next, immunization and generation of monoclonal antibodies to Compound B-KLH and Compound C-KLH is conducted.

### Synthesis of Compound C

### i. Preparation of Methyl 2-Methylpropionimidate Hydrochloride

(a) A water bath was placed on a 16-kg balance. A 4-L Erlenmeyer flask was placed in the water bath and fitted with a thermometer and a gas inlet tube. A mechanical stirring apparatus was suspended over the flask with sufficient agitator clearance to enable the addition of materials to the flask. Methyl tert-butyl ether (3.0 L) was added to the flask and ice water was added to the water bath. The stirrer was started and set for moderate agitation.

(b)The balance was tared. To the methyl tert-butyl ether was charged, through the gas inlet tube, anhydrous hydrogen chloride gas (420 g) over one hour. The rate of addition was controlled to maintain the batch temperature below 20° C.

(c) After the addition of anhydrous hydrogen chloride gas was complete, the solution was cooled to 5° C.

(d) Methanol (387.4 g) and isobutyronitrile (717.0 g) were combined and mixed for five minutes to obtain a uniform solution.

(e) The solution prepared in (d) was added dropwise to the batch, at a rate to maintain the batch temperature below 10° C. The resulting clear solution was allowed to warm to room temperature (23° C.) with continued stirring overnight. During the overnight stir, the product crystallized from the solution.

(f) The thick, white suspension was cooled in an ice/water bath to below 5° C. and stirred for approximately two hours.

(g) A 4-L filtration flask attached to a Gast® vane vacuum pump was assembled with a 253-mm Büchner fennel containing Sharkskin™ filter paper. The vacuum pump was started, the filter paper was wetted with methyl tert-butyl ether (100 mL) and pressed in place. After confirming a complete seal of the filter paper, the suspension from Step (f) was vacuum filtered and washed with methyl tert-butyl ether (500 mL).

(h)The first crop of product, methyl 2-methylpropionimidate hydrochloride, was air dried, weighed, transferred to a polyethylene bag, and stored at room temperature.

(i) The filtration from Step (g) was stored, without stirring, in a freezer (−10°C.) for three days. During the storage, the product crystallized from the solution.

(j) A 4-L filtration flask attached to a Gast® vane vacuum pump was assembled with a 150-mm Büchner funnel containing Sharkskin™ filter paper. The vacuum pump was started, the filter paper was wetted with methyl tert-butyl ether (100 mL) and pressed in place. After confirming a complete seal of the filter paper, the suspension from (i) was vacuum filtered and washed with methyl tert-butyl ether (200 mL).

(k) The second crop of product, methyl 2-methylpropionimidate hydrochloride, was air dried, weighed, transferred to a polyethylene bag, and stored at room temperature.

(l) The filter from (6) was stored, without stirring, in a freezer (−10°C.) for two weeks. During the storage, the product crystallized from the solution.

(m) A 4-L filtration flask attached to a Gast( vane vacuum pump was assembled with a 150-mm Büchner fennel containing Sharkskin™filter paper. The vacuum pump was started, the filter paper was wetted with methyl tert-butyl ether (100 mL) and pressed in place. After confirming a complete seal of the filter paper, the suspension from (l) was vacuum filtered and washed with methyl tert-butyl ether (200 mL ).

(n) The third crop of product, methyl 2-methylpropionimidate hydrochloride, was air dried, weighed, transferred to a polyethylene bag, and stored at room temperature.

### (ii) Preparation of 2-Methylpropion-N-cyanoimidate

(a) To a 25-L, polypropylene pail equipped with an overhead, mechanical stirrer and thermometer was charged methyl 2-methylpropionimidate hydrochloride (1507.0 g) and cold (approximately 2° C.) cyanamide, 50% aqueous solution (111.8 g).

(b) The stirrer was started and set a high speed to obtain a vigorous agitation. Sodium hydrogen phosphate dodecahydrate (5910.0 g) was added, in 200-300 g portions, over 2.25 hours. The pH of the reaction mixture was monitored with pH paper. The pH range was maintained between 5-6 during the sodium hydrogen phosphate dodecahydrate addition by the addition of water (4×300.0 mL), in portions.

(c) After the addition was completed, the slurry was stirred for one hour. The slurry thickened. Water (500 mL) and methyl tert-butyl ether (1.0 L) were added and the slurry was stirred for an additional two hours.

(d) A filtration apparatus attached to a Gast® vane vacuum pump was assembled with an 18.6-cm Büchner funnel containing Sharkskin™ filter paper. The filter paper was wetted with methyl tert-butyl ether (100.0 mL) and pressed in place. After confirming a complete seal of the filter paper, the solution from (c) was vacuum filtered and the filter cake was washed with methyl tert-butyl ether (2×500.0 mL).

(e) The filtrate was transferred to a 5-L separatory funnel and allowed to separate for approximately ten minutes. The layers were separated. The lower (aqueous) layer was transferred to a second 5-L separatory funnel. The upper (organic) layer was drained into an appropriate container and set aside for later use.

(f) The aqueous layer, from (e) was washed with methyl tert-butyl ether (500.0 mL) and the biphasic mixture was allowed to separate for approximately ten minutes. The lower (aqueous) layer was drained into the appropriate container and discarded.

(g) The organic phase from (f) was combined the organic phase from (e)

(h) Sodium sulfate (100.0 g) was added to the combined organic phases. This mixture was gently stirred, with a glass stirring rod, intermittently, for approximately 30 minutes.

(i) A filtration apparatus attached to a Gast® vane vacuum pump was assembled with a filter funnel containing Sharkskin™filter paper. The filter paper was wetted with methyl tert-butyl ether (100.0 IL) and pressed in place. After confirming a complete seal of the filter paper, the solution from (h) was vacuum filtered and the filter cake was washed with methyl tert-butyl ether (250.0 mL).

(j) The combined filtrate and the washings were transferred in portions to a 3-L rotary evaporator bulb that was connected to a rotary evaporator. The entire solution containing the product was concentrated under vacuum (approximately 23 to 26 inches Hg vacuum) to a constant weight. The product, methyl -methylpropion-N-cyanoimidate, was used directly in the next step.

### (iii) Preparation of 2-Metbylpropion-N-cyanometylamidine

(a) A four-neck, 5-L, round bottom flask was equipped with an overhead mechanical stirrer, a gas dispersion tube, a 500-mL addition funnel with a pressure equalizing arm, and a thermometer. The apparatus was placed in an empty water bath and charged with methanol (300 imL). The stirrer was started and nitrogen gas was bubbled through the solution for 30 minutes.

(b) The reactor was charged with methyl 2-methylpropion-N-cyanoimidate (264.6 g) and aminoacetonitrile hydrochloride (317.8 g). The reaction mixture was stirred and the bubbling of nitrogen was continued for 15 minutes.

(c) Triethylamine (408.4 g) was charged to the dropping funnel. The empty water bath was filled with ice/water and the reaction mixture was cooled to below 20° C.

(d)The triethylamine (408.4 g) was added at a rate to maintain the batch temperature below 40° C. The mixture was stirred at room temperature for approximately 12 hours after the 20 addition was complete. The reaction completion was checked by TLC analysis.

(e) A 4-L polypropylene beaker was placed into an empty water bath, fitted with a mechanical stirrer and charged with water (2.0 L). The stirrer was started and set a high speed to obtain a vigorous agitation. The contents of the flask from (d) were poured into the water, with continued stirring.

(f) An ice/water mixture was added to the water bath and cooling with vigorous stirring was maintained for approximately 1.5 hours. During this time, the product crystallized as a cream-colored solid.

(g) A filtration apparatus attached to a Gast® vane vacuum pump was assembled with an 18.6-cm Büchner funnel containing Sharkskin™ filter paper. The filter was wetted with water (100 mL) and pressed in place. After confirming a complete seal of the filter paper, the solution from (f) was filtered and the filter cake was washed with ice cold water (300 mL).

(h) The filter cake was air dried on the filter for approximately two hours (until the dripping of the filtrate into the receiver was negligible.).

(i) The solid was placed onto tared, glass drying pans and transferred to a drying oven. The solid was vacuum dried to constant weight using a high-vacuum pump (29 inches Hg vacuum) at 55° C. for approximately 72 hours.

(j) The dried compound, 2-methylpropion-N-cyano-N′-cyanomethylamidine, was removed from the oven, weighed, and transferred to an amber glass bottle for storage at ambient temperature.

### (iv) Preparation of 4-Amino-5-cyano-2-(2-mehylethyl)imidazole

(a) A 5-L, three-neck, round-bottom flask was fitted with a mechanical stirrer, nitrogen inlet, and a thermometer. Methyl 2-methylpropio-N-cyano-N′-cyanomethylamidine (450 g) and tetrahydrofuran (2.0 L) were charged to the flask and the stirrer was started. The mixture was stirred for 30 minutes at ambient temperature.

(b) To this heterogenous mixture, potassium tert-butoxide (673 g) was added in portions over two hours. The temperature was maintained between 30° C. and 40° C. by controlling the addition rate. At a temperature of 30° C., a homogeneous solution was obtained.

(c) After the addition of potassium tert-butoxide, the pale brown solution was stirred for an additional ten minutes at 40° C. The reaction completion was monitored by thin layer chromatography (TLC).

(d) To this reaction mixture, methanol (500 mL) was added and the solution was cooled to 5° C. using an ice bath.

(e) Concentrated hydrochloric acid (600 g of a 36% solution) was added over one hour at such a rate to maintain the batch temperature below 20° C. using an ice/water bath.

(f) A 4-L filtration apparatus attached to a Gast® vane vacuum pump was assembled with a 27-cm Büchner funnel containing filter paper. The filter paper was wetted with methanol (10 mL) and pressed in place. After confirming a complete seal of the filter paper, the solution from (e) was filtered and collected.

(g) The precipitate was then washed with methanol (2×500 mL) and the washings were collected.

(h) The filtrates from (f) and (g) were combined and transferred to a 3-L evaporator bulb that was connected to a rotary evaporator. The content of the bulb was concentrated to minimum volume under reduced pressure (approximately 25 inches Hg vacuum).

(i) Toluene (2.250 mL) was added to the 3-L evaporator bulb, and the contents of the bulb were further concentrated.

(j) To the 3-L evaporator bulb containing the clear, brown solution, ethyl acetate (1500-L) was added and the solution was heated at reflux for one hour.

(k) A 4-L filtration apparatus attached to Gast® vane vacuum pump was assembled with a 10-cm Büchner funnel containing filter paper. The filter paper was wetted with ethyl acetate (10 mL) and pressed in place. After confirming a complete seal of the filter paper, the hot ethyl acetate mixture from (j) was clarified and the filtrate was collected.

(l) The filtrate was stored in the refrigerator (5° C.) overnight, and the product precipitated.

(m) A 2-L filtration apparatus attached to a Gast® vane vacuum pump was assembled with a 24-cm Büchner funnel containing filter paper. The filter paper was wetted with ethyl acetate (10 mL) and pressed in place. After confirming a complete seal of the filter paper, the suspension from (1) was filtered.

(n) The product, 4-amino-5-cyano-2-(2-methylethyl)imidazole, was washed with cold (5° C.) ethyl acetate (250 mL) and air-dried.

(o) The filtrate from (m) was transferred to a 3-L evaporator bulb that was connected to a rotary evaporator. The contents of the evaporator bulb were concentrated to approximately 500 mL under reduced pressure (27 inches Hg vacuum).

(p) The rotary evaporator bulb and its contents were placed in an ice bath for five hours, and additional product crystallized.

(q) A 1-L filtration apparatus attached to a Gast( vane vacuum pump was assembled with a 10-cm Büchner funnel containing filter paper. The filter paper was wetted with ethyl acetate (10 mL) and pressed in place. After confirming a compete seal of the filtered paper, the solution from (p) was filtered.

(r) The product, 4-amino-5-cyano-2-(2-methylethyl)imidazole, was washed with cold ethyl acetate (100 mL).

(s) The wet first and second crops (from (n) and (r), respectively) were combined and dried at 60° C. overnight in a vacuum drying oven.

### (v) Preparation of 4-Amino-2-(2-methylethyl)imidazole-5-carboxamide

(a) A three-neck, 2-L, round bottom flask was placed in an electric heating mantle, fitted with an overhead mechanical stirrer, additional funnel, and a thermometer. Sulfuric acid, 96.3% (152.6 g) was charged to the flask and the stirrer was started.

(b) Water (10.8 g) was added to the dropping funnel. The water was added dropwise to the stirring acid. The mixture was stirred approximately five minutes after the addition was complete to ensure homogeneity.

(c) 4-Amino-5-cyano-2-(2-methylethyl)imidazole (131.6 g) was added in portions until the temperature reached approximately 90° C. A slower addition was continued at such a rate to maintain the batch temperature above 90° C. but not to exceed 100° C. The addition time was approximately two hours. After the addition was complete, the temperature was maintained between 90° C. and 100° C. by gentle heating for 30 minutes. The batch was then assayed for completeness of reaction by TLC.

(d) The three-neck, 2-L, round-bottom flask containing the thick reaction mixture was removed from the heating mantle and placed in a water bath. The bath was filled with an ice water mixture and the stirred reaction mixture was cooled to approximately 40° C.

(e) To this reaction mixture, methanol (250 mL) was cautiously added with efficient stirring at such a rate to maintain the batch temperature below 40° C. with ice bath cooling.

(f) Upon complete addition, the reaction mixture was stirred with ice bath cooling below 20° C. The stirrer was set to stir rapidly and sodium bicarbonate (450.0 g) was added in portions over three hours.

(g) To the thick slurry was added water (250.0 mL) and the mixture was stirred for one hour until a granular precipitate formed.

(h) A 20-cm Büchner funnel was assembled with a 2-L filtration flask receiver and the apparatus was attached to a Gast® vane vacuum pump. Filter paper was placed in the funnel and the vacuum pump was turned on. The filter paper was wetted with methanol (20 mL) and pressed in place. After confirming a complete seal of the filter paper, the suspension was vacuum filtered.

(i) The filter cake was washed with methanol (250 mL). The filter cake was manually compacted to minimize cracking and suction was maintained until very little filtrate was observed dripping into the receiver.

(j) The filter was transferred to a 3-L, round-bottom flask, placed on a rotary evaporator, and concentrated to a volume of approximately 500 mL.

(k) The flask was removed from the evaporator and placed in an ice water cooling bath. The flask was fitted with an overhead mechanical stirrer and the contents were stirred with cooling to 5° C. for about four hours.

(l) A 14-cm Büuchner funnel was assembled with a 2-L filtration flask receiver and the apparatus was attached to a Gasto vane vacuum pump. Filter paper was placed in the funnel and the vacuum pump was turned on. The filter paper was wetted with methanol (20 mL) and pressed in place. After confirming a complete seal of the filter paper, the suspension was vacuum filtered.

(m) The filter cake was placed in a tared, glass drying pan and dried at 60° C. in a vacuum oven (30 inches Hg vacuum) for approximately 15 hours.

(n) The dried product, 4-amino-2-(2-methylethyl)imidazole-5-carboxamide, was removed from the oven, weighed, transferred to a polyethylene bag, and stored at ambient temperature.

(o) The filtrate from (1) was transferred to a 1-L, round bottom flask, placed on a rotary evaporator, and concentrated to a volume of approximately 100 ML.

(p) A 10-cm Büchner funnel was assembled with a 2-L filtration flask receiver and the apparatus was attached to a Gaste vane vacuum pump. Filter paper was placed in the funnel and the vacuum pump was turned on. The filter paper was wetted with methanol (20 mL) and pressed in place. After confirming a complete seal of the filter paper, the suspension was vacuum filtered.

(q) The filter cake was placed in a tared, glass drying pan and dried at 60° C. in a vacuum oven (30 inches Hg vacuum) overnight for 14 hours.

(r) The dried product, 4-amino-2-(2-methylethyl)imidazole-5-carboxamide, was removed from the oven, weighed, transferred to a polyethylene bag, and stored at ambient temperature.

(s) The dried products from (n) (First Crop) and (r) (Second Crop) were combined, weighed, transferred to a polyethylene bag, and stored at ambient temperature.

### (vi) Preparation of 4-(3-cyclopentyloxy-4-methoxybenzylamino)-2-(2-methylethyl)imidazole-5-carboxamide

(a) A three-neck, 250-mL, round-bottom flask was placed in an electric heating mantle and equipped with a mechanical stirrer and a thermometer. Methanol (60 1L) was added and stirring was initiated. 4-Amino-2-(2-methylethyl)imidazole-5-carboxamide (12.6 g), p-toluenesulfonic acid, monohydrate (0.14 g), and 3-cyclopentyloxy-4-methoxybenzaldehyde (17.6 g) in methanol (15 mL) were added sequentially and the suspension was stirred.

(b) The reaction mixture was stirred for one hour. The batch was then analyzed by TLC to monitor the consumption of 4-amino-2-(2-methylethyl)imidazole-5-carboxamide (starting material).

(c) The reaction flask was fitted with a distillation head and a water-cooled distillation condenser and receiving flask. The heating mantle was plugged into a Variac power transformer. The power transformer was set to approximately 30% power and turned on. The reaction was heated to the boiling point.

(d) Upon reaching the boiling point, the solvent began to distill out. Approximately 30 mL of solvent was removed by atmospheric distillation. The heating was stopped and the heating mantle was replaced with an empty water bath.

(e) A mixture of ice/water was added to the water bath. The mixture was cooled with stirring until the temperature dropped below 20° C. and the ice bath was removed.

(f) Toluene (30 ml) was added to the mixture. The flask was fitted with a powder additional funnel and sodium borohydride (5.96 g) was added as a suspension in ethanol (40 mL) over approximately ten minutes with stirring.

(g) The heating mantle was plugged into a Variac power transformer. The power transformer was set to approximately 30% power and turned on. The reaction mixture was heated to approximately 40° C.

(h) The reaction mixture was heated at approximately 40° C. for one hour. The progress of imine reduction was monitored by TLC.

(i) When the reaction was determined to be complete, the heating was stopped and the stirring mixture was allowed to cool to room temperature.

(j) The reaction mixture was poured into water (60 ml) and the aqueous solution was extracted with ethyl acetate (3×70 mL).

(k) The organic extracts were combined and treated with magnesium sulfate (10 g). The aqueous solution was discarded.

(l) A 5.3-cm Büchner funnel was assembled with a 500-mL filtration flask receiver and the apparatus was attached to a Gast® vane vacuum pump. Filter paper was placed in the funnel and the vacuum pump was turned on. The filter paper was wetted with ethyl acetate (5 ml) and pressed in place. After confirming a complete seal of the filter paper, the suspension was vacuum filtered. The drying agent was washed with ethyl acetate (approximately 20 mL).

(m) The solution was transferred to a 500 mL, one-neck, round-bottom flask, which was connected to a rotary evaporator. The solution was concentrated to a volume of approximately 60mL.

(n) The flask was removed from the rotary evaporator and placed in an empty water bath on a magnetic stirring plate. Ethyl acetate (30 mL) was added and the stirrer was started. Ice/water was added to the bath and the mixture was stirred with cooling for four hours.

(o) A 5.3-cm Büchner funnel was assembled with a 250-ml filtration flask receiver and the apparatus was attached to a Gast® vane vacuum pump. Filter paper was placed in the funnel and the vacuum pump was turned on. The filter paper was wetted with ethyl acetate (approximately 5 mL) and pressed in place. After confirming a complete seal of the filter paper, the suspension was vacuum filtered. The solid was washed withe ethyl acetate (approximately 10 mL). The filter cake was manually compacted to minimize cracking and suction was maintained until very little filtrate was observed dripping into the receiver.

(p) The filter cake was placed in a tared, glass drying pan and dried at 60° C. in a vacuum oven (2 mm Hg vacuum) for approximately 15 hours.

(q) The dried product 4-(3-cyclopentyloxy-4-methoxybenzylamino)-2-(2-methylethyl)imidazole-5-carboxamide, was removed from the oven, weighed, transferred to an amber glass bottle, and stored at ambient temperature.

### (vii) Preparation of 4-(3-cyclopenloxy-4-methoxybenzyl)-8-(2-tnethylethyl)

(a) A three-neck, 250-mL, round-bottom flask was placed in an electric heating mantle, on a magnetic stirring plate and fitted with a distillation head with a water cooled distillation condenser and a thermometer. A magnetic stirring bar was placed in the flask. 4-(3-Cyclopentyloxy-4-methoxybenzylamino)-2-(2methoxybenzylamino)-2-(2-methylethyl)imidazole-5-carboxamide (17.5 g), triethylorthoformate (12 mL), p-toluenesulfonic acid, monohydrate (approximately 50 mg), and toluene (50 mL) were charged to the flask and the stirrer was started.

(b) The heating mantle was plugged into a Variac power transformer. The power transformer was set to approximately 30% power and turned on).

(c) The heating continued and the temperature was allowed to increase until noticeable reflux of the mixture was attained.

(d) Reflux was maintained and the distillate was collected. During this period the internal temperature of the mixture rose to 110° C. The batch was then analyzed by TLC.

(e) The three-neck, 250 mL, round-bottom flask containing the reaction mixture was removed from the heating mantle and placed in a water bath. The bath was filled with an ice/water mixture and the reaction was cooled with stirring to approximately 5° C.

(f) The reaction mixture was stirred with continued cooling for approximately one hour.

(g) A 8.3-cm Büchner funnel was assembled with a 250-mL filtration flask receiver and the apparatus was attached to a Gast® vane vacuum pump. Filter paper was placed in the funnel and the vacuum pump was turned on. The filter paper was wetted with toluene (10 mL) and pressed in place. After confirming a complete seal of the filter paper, the suspension was vacuum filtered.

(h) The filter cake was washed with toluene (25 mL). The filter cake was manually compacted to minimize cracking and suction was maintained until very little filtrate was observed dripping into the receiver.

(i) The filter cake was placed in a tared, glass drying pan and dried at 60° C. in a vacuum oven (2 mm Hg vacuum) for approximately 15 hours.

(j) The dried product, 3-(cyclopentyloxy-4-methoxybenzyl)-8-(2-methylethyl) hypoxanthine, was removed from the oven, weighed, transferred to an amber glass bottle, and stored at ambient temperature.

(k) The filtrate from Step (h) was transferred to a 250-mL, single-neck round-bottom flask, placed on a rotary evaporator, and concentrated to a volume of approximately 20 mL.

(l) The flask was removed from the evaporator, treated with ethyl acetate (30 mL) and placed in an ice/water cooling bath. The contents were magnetically stirred, with cooling to below 5° C. for about two hours.

(m) A 4.3-cm Büchner funnel was assembled with a 125-ml filtration flask receiver and the apparatus was attached to a Gasto vane vacuum pump. Filter paper was placed in the funnel and the vacuum pump was turned on. The filter paper was wetted with ethyl acetate (2 mL) and pressed in place. After confirming a complete seal of the filter paper, the suspension was vacuum filtered.

(n) The filter cake was washed with ethyl acetate (10 mL). The filter cake was manually compacted to minimize cracking and suction was maintained until very little filtrate was observed dripping into the receiver.

(o) The filter cake was placed in a tared, glass drying pan and dried at 60° C. in a vacuum oven (2 mm Hg vacuum) for approximately 15 hours.

(p) The dried product, 3-(cyclopentyloxy-4-methoxybenzyl)-8-(2-methylethyl)-hypoxanthine, was removed from the oven, weighed, transferred to an amber glass bottle, and stored at ambient temperature.

### (viii) Preparation of 3-(3-cyclopeintyloxy-4-methoxybenzyl)-8-isopropyl-3H-purine

Dry N,N-dimethylformamide (390 ml, 5.036 mol) was dissolved in dry dichloromethane (5 L) in a 20 L three necked flask and cooled to below 15° C. in an ice-water bath under nitrogen. Phosphorus oxychloride (475 ml, 5.096 mol) was added slowly maintaining the temperature below 15° C. during the addition. The hypoxanthine (1.5 Kg, 4.074 mol) was added in small portions maintaining the temperature below 15° C. and the resulting mixture stirred for 12-18h until TLC indicated the complete disappearance of starting material. The mixture was diluted with diisopropyl ether (5 L) and washed with water (2×3 L), the organic phase separated and the solvent removed under vacuum (less than 40° C.). To a 20L three necked flask, 0.88 ammonia solution (1.98 L) was added and the solution was cooled to below 5° C. using an ice-water bath. The solution of the crude chloropurine prepared earlier was diluted with dichloromethane (2 L) and added slowly to the ammonia solution, maintaining the temperature below 25° C. and the resulting mixture was then stirred for 24 hours. The complete disappearance of chloropurine intermediate was confirmed by TLC and when the reaction was completed, the reaction contents were added to IM sodium hydroxide solution (2 L) in a separating funnel and the organic layer was separated. The aqueous layer was extracted with dichloromethane (4 L). 6M hydrochloric acid (2 L) was added to the organic layer in a separating funnel and the organic layer was collected. The organic extract was treated with activated charcoal (20 g), the mixture filtered through celite and the filtrate concentrated to dryness in vacuo. The crude hydrochloride salt was recrystallised from acetonitrile to give the adenine Compound C as a white microcrystalline solid (yields of 50-80% were obtained for Compound A on this scale).

### Antibodies Specific for PDEIV Inhibitors

Antibodies recognizing the conjugated analogs are immobilized as described and used to construct an affinity column, using the method described in U.S. Pat. No. 5,316,932, herein incorporated by reference. Additional methods useful for coupling of antibodies to support matrices include Fluorescent Probes and Research Chemicals, Chapter 5, Crosslinking and photoreactive probes. Molecular Probes, Eugene, Oreg., 1998; Harlowe and Lane, Antibodies: A Laboratory Manual, Cold Spring Harbor Publications, N.Y. (1988); Purification of murine MHC antigens by monoclonal antibody affinity chromatography, Methods Enzymol. 1983;92:86-109; and Use of monoclonal antibody immunoaffinity columns to purify subsets of human HLA-DR antigens, Methods Enzymol. 1984;108:600-6, herein incorporated by reference.

The antibody affinity column is used to screen mixtures of compounds to identify novel compounds that belong to the same class of compounds as the two PDEIV inhibitor analogs.

Alternatively, the microtiter plate assay procedure may be used.

The antibodies specific for PDEIV inhibitor analogs can be used as described above for modeling the PDEIV binding site, or for generating structure-activity relationship for PDEIV inhibitors.

In addition, the antibodies may be used to generate anti-idiotype antibodies, which may then be used as a model for the design of additional PDEIV inhibitor compounds.

While there have been described what are presently believed to be the preferred embodiments of the invention, those skilled in the art will realize that changes and modifications may be made thereto without departing from the spirit of the invention. It is intended to claim all such changes and modifications that fall within the true scope of the invention.

What is claimed is:

1. A method of defining the portion of one or more chemical compounds having binding affinity for a target receptor comprising:
(a) identifying one or more key component fragments of one or more chemical compounds having binding affinity for a target receptor;
(b) coupling one or more analogs of the one or more chemical compounds to a carrier molecule to construct one or more analog-carrier conjugates, said analogs containing one or more of the key component fragments, said analog being coupled to the carrier such that one or more of the key component fragments are exposed;
(c) utilizing the analog-carrier conjugates to generate monoclonal antibodies in vivo or in vitro that are able to define the exposed key component fragments; and
(d) determining the monoclonal antibodies which are most specific for the key component fragments of the one or more chemical compounds and which bind to the one or more chemical compounds.

2. The method of claim 1 wherein the ability of the monoclonal antibodies to mimic the binding site of a target receptor is determined.

3. The method of claim 1, wherein two or more analogs are utilized to generate a panel of monoclonal antibodies, and wherein each analog-carrier conjugate defines a key component fragment of the one or more chemical compounds, and wherein the analog-carrier conjugates together define all of the key component fragments of the one or more chemical compounds.

4. The method of claim 3, wherein each member of the panel of monoclonal antibodies binds specifically to a portion of the one or more chemical compounds.

5. The method of claim 1, wherein two or more analogs are utilized to generate a panel of monoclonal antibodies, wherein two or more carrier conjugates defines a key component fragment of the one or more chemical compounds, and wherein the analog-carrier conjugates together define a portion of the entire surface conformation of the one or more chemical compounds.

6. The method of claim 1, wherein the monoclonal antibodies generated for each analog are tested for their dissociation constant, and those exhibiting the strongest binding are included in a panel such that each different analog is represented by monoclonal antibodies in the panel.

7. The method of claim 6, wherein the dissociation constant is in the range of from about 0.01 nM to about 10 nM.

8. The method of claim 1, wherein the monoclonal antibodies are generated using in vivo immunization methods.

9. The method of claim 1, wherein the monoclonal antibodies are generated using in vitro immunization methods.

10. The method of claim 1, wherein the carrier molecule is selected from the group consisting of Keyhole Limpet Hemocyanin, ovalbumin and thyroglobulin.

11. The method of claim 1, wherein the one or more chemical compounds exhibit PDEIV inhibitor or opiate activity.

12. The method of claim 1, wherein the functional groups are selected from the group consisting of carboxyl, hydroxyl, keto, amino, nitro, and sulfhydryl.

13. The method of claim 1, wherein the one or more chemical compounds identified are organic molecules.

14. The method of claim 1, wherein the one or more chemical compounds identified are inorganic molecules.

15. The method of claim 1, wherein the one or more chemical compounds identified are biological molecules.

16. The method of claim 1, wherein the one or more chemical compounds having binding affinity for a target receptor have a molecular weight of less than approximately 1000 g/mole.

17. The method of claim 1, wherein the one or more chemical compounds having binding affinity for a target receptor have a molecular weight of less than approximately 500 g/mole.

18. The method of claim 1, wherein the panel of monoclonal antibodies is comprised of 2-3 monoclonal antibodies.

19. The method of claim 1, wherein the analogs either (i) contain at least one functional group allowing attachment of the analog to the carrier molecule; or (ii) are modified to contain at least one functional group allowing attachment of the analog to the carrier molecule.

20. The method of claim 1, further comprising verifying that additional small molecules are part of the structural class to which the compound that generically defines the surface conformation and surface charge density belongs.

21. The method of claim 1, wherein a three-dimensional structure of the antigen binding region of one or more of the monoclonal antibodies is determined.

22. The method of claim 21, wherein the three-dimensional structure of the antigen binding region is used to design new molecules.

23. The method of claim 21, wherein the three-dimensional structure of the antigen binding region is used to evaluate the exterior molecular characteristics of one or more compounds of interest to determine whether the compounds of interest have binding affinity for the target receptor.

24. A method of identifying compounds which have binding affinity for a target receptor comprising:
(a) identifying one or more key component fragments of one or more chemical compounds having binding affinity for a target receptor;
(b) coupling one or more analogs of the one or more chemical compounds to a carrier molecule to construct one or more analog-carrier conjugates, said analogs containing one or more of the key component fragments, said analog being coupled to the carrier such that one or more of the key component fragments are exposed;
(c) utilizing the analog-carrier conjugates to generate monoclonal antibodies in vivo or in vitro that are able to define the exposed key component fragments; and
(d) determining the monoclonal antibodies which are most specific for the key component fragments of the one or more chemical compounds and which bind to the one or more chemical compounds;
(e) immobilizing the monoclonal antibodies on a support; and
(f) conducting a series of assays utilizing said immobilized monoclonal antibodies to screen one or more compounds of interest.

25. The method of claim 24, wherein the ability of the monoclonal antibodies to mimic the binding site of a target receptor is determined.

26. The method of claim 24, wherein two or more analogs are utilized to generate a panel of monoclonal antibodies, and wherein, each analog-carrier conjugate defines a key component fragment of the one or more chemical compounds, and wherein the analog-carrier conjugates together define all of the key component fragments of the one or more chemical compounds.

27. The method of claim 24, wherein two or more analogs are utilized to generate a panel of monoclonal antibodies, and wherein each analog-carrier conjugate defines a key component fragment of the one or more chemical compounds, and wherein the analog-carrier conjugates together define a portion of the entire surface conformation of the one or more chemical compounds.

28. The method of claim 24, wherein the monoclonal antibodies generated for each analog are tested for their dissociation constant and those exhibiting the strongest binding are included in a panel such that each different analog is represented by monoclonal antibodies in the panel.

29. The method of claim 28, wherein the dissociation constant is in the range from about 0.01 nM to about 10 nM.

30. The method of claim 24, wherein the monoclonal antibodies are generated using in vivo immunization methods.

31. The method of claim 24, wherein the monoclonal antibodies are generated using in vitro immunization methods.

32. The method of claim 24, wherein the functional groups are selected from the group consisting of carboxyl, hydroxyl, keto, amino, nitro, and sulfhydryl.

33. The method of claim 24, wherein the one or more compounds of interest are obtained from 1) combinatorial libraries of peptides, synthetic ethers, or phosphonates; 2) natural product extracts; 3) microbial or other cell culture broths; 4) synthetic products; 5) synthetic analogs; 6) intermediates of drug candidates or drug metabolites.

34. The method of claim 24, wherein the carrier molecule is selected from the group consisting of Keyhole Limpet Hemocyanin, ovalbumin and thyroglobulin.

35. The method of claim 24, wherein the one or more chemical compounds exhibit PDEIV inhibitor or opiate activity.

36. The method of claim 24, wherein the panel of monoclonal antibodies is pooled before attachment to the support.

37. The method of claim 24, wherein each member of the panel of monoclonal antibodies is utilized separately to screen compounds of interest.

38. The method of claim 24, wherein the one or more chemical compounds are organic molecules.

39. The method of claim 24, wherein the one or more chemical compounds having binding affinity for a target receptor have a molecular weight of less than approximately 1000 g/mole.

40. The method of claim 24, wherein the one or more chemical compounds having binding affinity for a target receptor have a molecular weight of less than approximately 500 g/mole.

41. The method of claim 24, wherein the one or more chemical compounds identified are inorganic molecules.

42. The method of claim 24, wherein the one or more chemical compounds identified are biological molecules.

43. The method of claim 24, wherein the panel of monoclonal antibodies is comprised of 2-3 monoclonal antibodies.

44. A method of defining a portion of one or more PDEIV inhibitors comprising:
(a) identifying one or more key component fragments of one or more PDEIV inhibitors;
(b) coupling one or more analogs of the one or more PDEIV inhibitors to construct one or more analog-carrier conjugates, said analogs containing one or more of the key component fragments, said analogs being coupled to the carrier such that one or more of the key component fragments are exposed;
(c) utilizing the analog-carrier conjugates to generate monoclonal antibodies in vivo or in vitro that define the exposed key component fragments; and
(d) determining the antibodies which are most specific for the key component fragments of the one or more PDEIV inhibitors and which bind to the one or more PDEIV inhibitors.

45. The method of claim 44, wherein the key component fragments are derived from 3-(3-cyclopentyloxy-4-methoxybenzyl)-6-ethylamino-8-isopropyl-3H-purine.

46. The method of claim 44, wherein the analogs that may be attached to the carrier molecule are 3-(3-cyclopentyloxy-4-methoxybenzyl)-6-amino-8-isopropyl-purine and 6-ethylamino-8-isopropyl-3-(p-aminomethlyphenyl)-purine.

47. The method of claim 44, wherein the analogs that may be attached to the carrier molecule are rolipram, 3-(3-cyclopentyloxy-4-methoxybenzyl)-6-ethylamino-8-isopropyl-purine or RP73401, wherein the analogs are attached via an ether linkage from the 4-O to the carrier molecule.

48. The method of claim 44, wherein the key component fragments are derived from 3,5-di-t-butyl-4-hydroxy-benzyl-5-chloro-2-(2-pyridyl)-ethynyl-benzoxoazole.

49. A method of identifying compounds which inhibit PDEIV comprising:
(a) identifying one or more key component fragments of one or more PDEIV inhibitors;
(b) coupling one or more analogs of the one or more PDEIV inhibitors to a carrier molecule to construct one or more analog-carrier conjugates, said analogs containing one or more of the key component fragments, said analog being coupled to the carrier such that one or more of the key component fragments are exposed;
(c) utilizing the analog-carrier conjugates to generate monoclonal antibodies in vivo or in vitro that are able to define the exposed key component fragments; and
(d) determining the monoclonal antibodies which are most specific for the key component fragments of the one or more chemical compounds and which bind to the one or more PDEIV inhibitors;
(e) immobilizing the monoclonal antibodies on a support; and
(f) conducting a series of assays utilizing said immobilized monoclonal antibodies to screen one or more compounds of interest.

50. The method of claim 49, wherein the key component fragments are derived from 3-(3-cyclopentyloxy-4-methoxybenzyl)-6-ethylamino-8-isopropyl-purine.

51. The method of claim 49, wherein the analogs that may be attached to the carrier molecule are 3-(3-cyclopentyloxy-4-methoxybenzyl)-6-amino-8-isopropyl-purine and 6-ethylamino-8-isopropyl-3-(p-aminomethlyphenyl)-purine.

52. The method of claim 49, wherein the analogs that may be attached to the carrier are rolipram, 3-(3-cyclopentyloxy-4-methoxybenzyl)-6-ethylamino-8-isopropyl-purine or RP73401, wherein the analogs are attached via an ether linkage from the 4-0 to the carrier molecule.

53. The method of claim 49, wherein the key component fragments are derived from 3,5-di-t-butyl-4-hydroxy-benzyl-5-chloro-2-(2-pyridyl)-ethynyl-benzoxoazole.

54. A method of designing molecules possessing defined exterior molecular characteristics comprising:
(a) identifying one or more key component fragments of one or more chemical compounds having binding affinity for a target receptor;
(b) coupling one or more analogs of the one or more chemical compounds to a carrier molecule to construct one or more analog-carrier, said analogs containing one or more of the key component fragments, said analog being coupled to the carrier such that one or more of the key component fragments are exposed;
(c) utilizing the analog-carrier conjugates to generate monoclonal antibodies in vivo or in vitro that are able to define the exposed key component fragments; and
(d) determining the monoclonal antibodies which are most specific for the key component fragments of the one or more chemical compounds and which bind to the one or more chemical compounds;
(e) immunizing an animal with the monoclonal antibodies to generate anti-idiotype monoclonal antibodies reactive against the antigen binding region of the immunizing monoclonal antibodies; and
(f) determining a three-dimensional structure of the antigen binding region of the anti-idiotypic antibodies.

55. The method of claim 54, wherein the three-dimensional structure of the antigen binding region is used to design new molecules.

56. The method of claim 54, wherein the three-dimensional structure of the antigen binding region is used to evaluate the exterior molecular characteristics of one or more compounds of interest to determine whether the compounds of interest have binding affinity for the target receptor.

57. The method of claim 54, further comprising testing the anti-idiotype monoclonal antibodies to determine whether the anti-idiotype monoclonal antibodies bind to the target receptor.

58. The method of claim 58, further comprising generating anti-anti-idiotypic antibodies with binding specificity for the anti-idiotypic monoclonal antibody antigen binding region.

59. The method of claim 58, further comprising determining the ability of the anti-anti-idiotypic monoclonal antibody to bind to the one or more chemical compounds having binding affinity for the target receptor.

60. The method of claim 54, wherein the carrier molecule is selected from the group consisting of Keyhole Limpet Hemocyanin, ovalbumin and thyroglobulin.

61. The method of claim 54, wherein the one or more chemical compounds exhibits PDEIV inhibitor or opiate activity.

62. The method of claim 54, wherein the functional groups are selected from the group consisting of carboxyl, hydroxyl, keto, amino, nitro, and sulfhydryl.

63. The method of claim 44 wherein the ability of the monoclonal antibodies to mimic the binding site of PDEIV is determined.

64. The method of claim 44, wherein two or more analogs are utilized to generate a panel of monoclonal antibodies, and wherein each analog-carrier conjugate defines a key component fragment of the one or more PDEIV inhibitors, wherein the analog-carrier conjugates together define all of the key component fragments of the one or more PDEIV inhibitors.

65. The method of claim 64, wherein each member of the panel of monoclonal antibodies binds specifically to a portion of the one or more PDEIV inhibitors.

66. The method of claim 44, wherein two or more analogs are utilized to generate a panel of monoclonal antibodies, wherein two or more carrier conjugates defines a key component fragment of the one or more PDEIV inhibitors, and wherein the analog-carrier conjugates together define a portion of the entire surface conformation of the one or more PDEIV inhibitors.

67. The method of claim 44, wherein the monoclonal antibodies generated for each analog are tested for their dissociation constant, and those exhibiting the strongest binding are included in a panel such that each different analog is represented by monoclonal antibodies in the F panel.

68. The method of claim 67, wherein the dissociation constant is in the range of from about 0.01 nM to about 10 nM.

69. The method of claim 44, wherein the monoclonal antibodies are generated using in vivo immunization methods.

70. The method of claim 44, wherein the monoclonal antibodies are generated using in vitro immunization methods.

71. The method of claim 44, further comprising verifying that additional small molecules are part of the structural class to which the compound that generically defines the surface conformation and surface charge density belongs.

72. The method of claim 44, wherein a three-dimensional structure of the antigen binding region of one or more of the monoclonal antibodies is determined.

73. The method of claim 72, wherein the three-dimensional structure of the antigen binding region is used to design new molecules.

74. The method of claim 72, wherein the three-dimensional structure of the antigen binding region is used to evaluate the exterior molecular characteristics of one or more compounds of interest to determine whether the compounds of interest have binding affinity for the target receptor.

75. The method of claim 1, wherein the carrier molecule is selected from the group consisting of Keyhole Limpet Hemocyanin, ovalbumin and thyroglobulin.

76. The method of claim 1, wherein the functional groups are selected from the group consisting of carboxyl, hydroxyl, keto, amino, nitro, and sulfhydryl.

77. The method of claim 1, wherein the panel of monoclonal antibodies is comprised of 2-3 monoclonal antibodies.

78. The method of claim 1, wherein the analogs either (i) contain at least one functional group allowing attachment of the analog to the carrier molecule; or (ii) are modified to contain at least one functional group allowing attachment of the analog to the carrier molecule.

79. The method of claim 49, wherein the ability of the monoclonal antibodies to mimic the binding site of PDEIV is determined.

80. The method of claim 49, wherein two or more analogs are utilized to generate a panel of monoclonal antibodies, and wherein, each analog-carrier conjugate defines a key component fragment of the one or more PDEIV inhibitors, and wherein the analog-carrier conjugates together define all of the key component fragments of the PDEIV inhibitors.

81. The method of claim 49, wherein two or more analogs are utilized to generate a panel of monoclonal antibodies, and wherein each analog-carrier conjugate defines a key component fragment of the one or more PDEIV inhibitors, and wherein the analog-carrier conjugates together define a portion of the entire surface conformation of the one or more PDEIV inhibitors.

82. The method of claim 49, wherein the monoclonal antibodies generated for each analog are tested for their dissociation constant and those exhibiting the strongest binding are included in a panel such that each different analog is represented by monoclonal antibodies in the panel.

83. The method of claim 82, wherein the dissociation constant is in the range from about 0.01 nM to about 10 nM.

84. The method of claim 49, wherein the monoclonal antibodies are generated using in vivo immunization methods.

85. The method of claim 49, wherein the monoclonal antibodies are generated using in vitro immunization methods.

86. The method of claim 49, wherein the functional groups are selected from the group consisting of carboxyl, hydroxyl, keto, amino, nitro, and sulfhydryl.

87. The method of claim 49, wherein the one or more compounds of interest are obtained from 1) combinatorial libraries of peptides, synthetic ethers, carbohydrates or phosphonates; 2) natural product extracts; 3) microbial or other cell culture broths; 4) synthetic products; 5) synthetic analogs; 6) intermediates of drug candidates or drug metabolites.

88. The method of claim 49, wherein the carrier molecule is selected from the group consisting of Keyhole Limpet Hemocyanin, ovalbumin and thyroglobulin.

89. The method of claim 49, wherein the panel of monoclonal antibodies is pooled before attachment to the support.

90. The method of claim 49, wherein each member of the panel of monoclonal antibodies is utilized separately to screen compounds of interest.

91. The method of claim 49, wherein the panel of monoclonal antibodies is comprised of 2-3 monoclonal antibodies.
